(19) United States
(12) Patent Application Publication
(10) Pub. No.: US 2011/0301209 A1
(43) Pub. Date: Dec. 8, 2011

(54) METHODS AND COMPOSITIONS FOR THE TREATMENT OF CANCER, TUMORS, AND TUMOR-RELATED DISORDERS

(75) Inventors: Sara L. Zaknoen, Carlsbad, CA (US); Tracy Lawhon, Encinitas, CA (US)

(73) Assignee: TRAGARA PHARMACEUTICALS, INC., San Diego, CA (US)

(21) Appl. No.: 13/132,901

(22) PCT Filed: Nov. 24, 2008
(86) PCT No.: PCT/US08/84586

§ 371 Date: Aug. 18, 2011

Related U.S. Application Data
(60) Provisional application No. 60/990,900, filed on Nov. 28, 2007.
(60) Provisional application No. 61/044,425, filed on Apr. 11, 2008.

(51) Int. Cl. A61K 31/427; A61P 35/00; A61P 35/02; A61K 31/402

(52) U.S. Cl. 514/365; 514/427

(57) ABSTRACT

Described herein are compositions and methods for using these compositions in the treatment of cancer, tumors, and tumor-related disorders in a subject.

### CROSS-REFERENCE

This application claims the benefit of U.S. Provisional Application No. 60/990,900, filed Nov. 28, 2007; and U.S. Provisional Application No. 61/044,425, filed Apr. 11, 2008, the contents of which are incorporated herein by reference in their entirety.

### FIELD

The present invention relates to combination compositions and the use of such combinations for the treatment of cancer, tumors, and tumor-related disorders.

### BACKGROUND

Cancer, tumors, tumor-related disorders, and neoplastic disease states are serious and oftentimes life-threatening conditions. These diseases and disorders, which are characterized by rapidly-proliferating cell growth, continue to be the subject of research efforts directed toward the identification of therapeutic agents which are effective in the treatment thereof. Such agents prolong the survival of the patient, inhibit the rapidly-proliferating cell growth associated with the neoplasm, or effect a regression of the neoplasm.

Generally, surgery and radiation therapy are the first modalities considered for the treatment of cancer that is considered locally confined, and offer the best prognosis. Chemotherapy treatment of certain cancers typically results in disappointing survival rates but still offer a survival benefit. For example, in patients with non-small cell lung cancer, platinum-based chemotherapy regimens, such as the use of either cisplatin or carboplatin plus one of either paclitaxel, docetaxel, gemcitabine, vinorelbine, irinotecan, etoposide, vinblastine, or bevacizumab is employed. If patients cannot tolerate this therapy, a single agent, such as N-(3-ethynylphenyl)-6,7-bis(2-methoxyethoxy)quinazolin-4-amine, commonly known as erlotinib (Tarceva®), can be used. Erlotinib targets the epidermal growth factor receptor tyrosine kinase which is highly expressed and occasionally mutated in various forms of cancer. If patients fail to respond to an erlotinib treatment, additional conventional treatment offers limited benefit.

Despite erlotinib's approval for the treatment of stage IIIB and IV non-small cell lung cancer, as with most therapeutic agents, side-effects result from its use. For example, common side effects, occurring in greater than 30% of patients taking erlotinib, include, rash, diarrhea, poor appetite, fatigue, shortness of breath, cough, nausea and vomiting. Additionally, less common side effects include infection, mouth sores, itching, dry skin, eye irritation, pulmonary fibrosis, and abdominal pain. Of greater concern, is the growing view that, while utilization of erlotinib for the treatment of tumors may initially shrink the size of the tumor, the tumor may eventually enlarge in size, indicating, among other things, the development of resistance. Erlotinib may be representative of the types of therapeutic agents being used for cancer treatment; in that its use has an effect on cancer, but because of other factors, which are not entirely known, the tumor develops resistance and progresses.

What is needed, therefore, are compositions and/or methods of treatment for cancer which take advantage of the synergy found in a therapeutic combination that could increase the effectiveness of the agents and reduce and/or eliminate the side effects typically associated with conventional treatments.

### SUMMARY

In one embodiment is a method for treating a subject having cancer, comprising administering to the subject, a therapeutically effective amount of a combination comprising a 1,2-diphenylpyrrole derivative or the respective pharmaceutically acceptable salt, solvate, polymorph or prodrug and a microtubule inhibitor.

In another embodiment is the method wherein the 1,2-diphenylpyrrole derivative has the following formula:

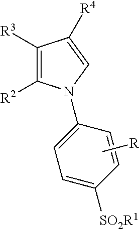

wherein:
R is a hydrogen atom, a halogen atom or an alkyl group having from 1 to 6 carbon atoms;
R¹ is an alkyl group having from 1 to 6 carbon atoms or an amino group;
R² is a phenyl group which is unsubstituted or is substituted by at least one substituent selected from the group consisting of substituents α and substituents β;
R³ is a hydrogen atom, a halogen atom or an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms;
R⁴ is a hydrogen atom; an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms; a cycloalkyl group having from 3 to 8 carbon atoms, an aryl group; or an aralkyl group; said aryl group having from 6 to 14 ring carbon atoms in a carbocyclic ring and are unsubstituted or are substituted by at least one substituent selected from the group consisting of substituents α and substituents β;
said aralkyl group are an alkyl group having from 1 to 6 carbon atoms and which are substituted by at least one aryl group as defined above;
said substituents α are selected from the group consisting of a hydroxy group, a halogen atom, an alkoxy group having from 1 to 6 carbon atoms and an alkylthio group having from 1 to 6 carbon atoms;
said substituents β are selected from the group consisting of an alkyl group which has from 1 to 6 carbon atoms and which is unsubstituted or are substituted by at least one substituent selected from the group consisting of a hydroxy group, a halogen atom, an alkoxy group having from 1 to 6 carbon atoms and an alkylthio group having from 1 to 6 carbon atoms; an alkanoyloxy group having from 1 to 6 carbon atoms; a mercapto group; an alkanoylthio group having from 1 to 6 carbon atoms; an alkylsulfinyl group having from 1 to 6 carbon atoms; a cycloalkloxy group having from 3 to 8 carbon atoms; a haloalkoxy group having from 1 to 6 carbon atoms; and an alkylenedioxy group having from 1 to 6 carbon atoms; or a pharmaceutically acceptable salt, solvate, or prodrug.

In another embodiment is the method wherein:
R is a hydrogen atom, a halogen atom or an alkyl group having from 1 to 4 carbon atoms;
R¹ is a methyl group or an amino group;
R² is an unsubstituted phenyl group or a phenyl group which is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and which is substituted by at least one substituent selected from the group consisting of a halogen atom, an alkoxy group having from 1 to 4 carbon atoms and an alkylthio group having from 1 to 4 carbon atoms; a haloalkoxy group having from 1 to 4 carbon atoms; and an alkylenedioxy group having from 1 to 4 carbon atoms;
R³ is a hydrogen atom, a halogen atom, an unsubstituted alkyl group having from 1 to 4 carbon atoms or a substituted alkyl group having from 1 to 4 carbon atoms and substituted by at least one substituent selected from the group consisting of a halogen atom, an alkoxy group having from 1 to 4 carbon atoms and an alkylthio group having from 1 to 4 carbon atoms;
R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to carbon atoms; a cycloalkyl group having from 3 to 6 carbon atoms; an aryl group which has from 6 to 10 ring carbon atoms and which is unsubstituted or is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to 4 carbon atoms; and a cycloalkyloxy group having from 3 to 7 carbon atoms; an aralkyl group having from 1 to 4 carbon atoms in the alkyl part and containing at least one said aryl group; or a pharmaceutically acceptable salt, solvate, or prodrug.

In another embodiment is the method wherein:
R is a hydrogen atom;
R¹ is an amino group;
R² is an unsubstituted phenyl group or a phenyl group which is substituted by at least one substituent selected from the group consisting of a halogen atom, an alkoxy group having from 1 to 4 carbon atoms, an alkylthio group having from 1 to 4 carbon atoms, an alkyl group having from 1 to 4 carbon atoms, a haloalkyl group having from 1 to 4 carbon atoms, a haloalkoxy group having from 1 to 4 carbon atoms and a alkylenedioxy group having from 1 to 4 carbon atoms;
R³ is a hydrogen atom, a halogen atom, an alkyl group having from 1 to 4 carbon atoms or a haloalkyl group having from 1 to 4 carbon atoms;
R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group and an alkoxy group having from 1 to 4 carbon atoms; a cycloalkyl group having from 3 to 6 carbon atoms; an aryl group which has from 6 to 10 ring carbon atoms and which is unsubstituted or is substituted by at least one substituent selected from the group consisting of a hydroxy group; a halogen atom; an alkoxy group having from 1 to 4 carbon atoms; an unsubstituted alkyl group having from 1 to 4 carbon atoms; an alkyl group having from 1 to 4 carbon atoms and which is unsubstituted or substituted by at least one halogen atom; and a cycloalkyloxy group having from 3 to 7 carbon atoms; and an aralkyl group having from 1 to 4 carbon atoms in the alkyl part and containing at least one said aryl group; or a pharmaceutically acceptable salt, solvate, or prodrug.

In another embodiment is the method wherein the 1,2-diphenylpyrrole derivative is selected from the group consisting of 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole.

In another embodiment is the method wherein the 1,2-diphenylpyrrole derivative is 2-(4-ethoxyphenyl)-4-methyl-1-(4-sulfamoylphenyl)-pyrrole.

In another embodiment is the method wherein the microtubule inhibitor is selected from docetaxel, paclitaxel and ixabepilone.

In another embodiment is the method wherein the microtubule inhibitor is docetaxel.

In another embodiment is the method wherein the 1,2-diphenylpyrrole derivative is 2-(4-ethoxyphenyl)-4-methyl-1-(4-sulfamoylphenyl)-pyrrole and the microtubule inhibitor is docetaxel.

In another embodiment is the method wherein the 1,2-diphenylpyrrole derivative and the microtubule inhibitor are administered sequentially in either order or simultaneously.

In another embodiment is the method of claim 1 wherein the 1,2-diphenylpyrrole derivative is administered first.

In another embodiment is the method wherein the microtubule inhibitor is administered first.

In another embodiment is the method wherein administering the combination enhances treatment of the subject.

In another embodiment is the method wherein administering the combination reduces the side effects of the treatment of cancer compared to a treatment with the microtubule inhibitor alone or a treatment of the 1,2-diphenylpyrrole derivative alone.

In another embodiment is the method wherein administering the combination is through oral, parenteral, buccal, intranasal, epidural, sublingual, pulmonary, local, rectal, or transdermal administration.

In another embodiment is the method wherein administering the combination is through parenteral administration.

In another embodiment is the method wherein parenteral administration is intravenous, subcutaneous, intrathecal, or intramuscular.

In another embodiment is the method wherein the 1,2-diphenylpyrrole derivative is administered orally every day and the microtubule inhibitor is administered by injection with a frequency selected from once every day, once every other day, once every seven days, once every fourteen days, once every twenty-one days, and once every twenty-eight days per a treatment cycle.

In another embodiment is the method wherein the cancer is selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

In another embodiment is the method wherein the cancer is non-small cell lung cancer, pancreatic cancer, breast cancer, ovarian cancer, colorectal cancer, and head and neck cancer.

In another embodiment is the method wherein the cancer is a carcinoma, a tumor, a neoplasm, a lymphoma, a melanoma, a glioma, a sarcoma, and a blastoma.

In another embodiment is the method wherein the carcinoma is selected from the group consisting of carcinoma, adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiatied carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial, metastatic carcinoma, mucoepidermoid carcinoma, cholangiocarcinoma, actinic keratoses, cystadenoma, and hepatic adenomatosis.

In another embodiment is the method wherein the tumor is selected from the group consisting of astrocytic tumors, malignant mesothelial tumors, ovarian germ cell tumor, supratentorial primitive neuroectodermal tumors, Wilm's tumor, pituitary tumors, extragonadal germ cell tumor, gastrinoma, germ cell tumors, gestational trophoblastic tumor, brain tumors, pineal and supratentorial primitive neuroectodermal tumors, pituitary tumor, somatostatin-secreting tumor, endodermal sinus tumor, carcinoids, central cerebral astrocytoma, glucagonoma, hepatic adenoma, insulinoma, medulloepithelioma, plasmacytoma, vipoma, and pheochromocytoma.

In another embodiment is the method wherein the neoplasm is selected from the group consisting of: intaepithelial neoplasia, multiple myeloma/plasma cell neoplasm, plasma cell neoplasm, interepithelial squamous cell neoplasia, endometrial hyperplasia, focal nodular hyperplasia, hemangioendothelioma, and malignant thymoma.

In another embodiment is the method wherein the lymphoma is selected from the group consisting of: nervous system lymphoma, AIDS-related lymphoma, cutaneous T-cell lymphoma, non-Hodgkin's lymphoma, lymphoma, and Waldenstrom's macroglobulinemia.

In another embodiment is the method wherein the melanoma is selected from the group consisting of: acral lentiginous melanoma, superficial spreading melanoma, uveal melanoma, lentigo maligna melanomas, melanoma, intraocular melanoma, adenocarcinoma nodular melanoma, and hemangioma.

In another embodiment is the method wherein the sarcoma is selected from the group consisting of adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma.

In another embodiment is the method wherein the glioma is selected from the group consisting of: glioma, brain stem glioma, and hypothalamic and visual pathway glioma.

In another embodiment is the method wherein the blastoma is selected from the group consisting of: pulmonary blastoma, pleuropulmonary blastoma, retinoblastoma, neuroblastoma, medulloblastoma, glioblastoma, and hemangiblastomas.

In another embodiment is a method of inducing differentiation of tumor cells, the method comprising contacting the cells with an effective amount of a combination comprising a 1,2-diphenylpyrrole derivative and a microtubule inhibitor whereby the combination induces differentiation of tumor cells.

In another embodiment is a method of inhibiting proliferation of cancer cells, the method comprising contacting a cancer cell with a combination comprising a 1,2-diphenylpyrrole derivative and a microtubule inhibitor whereby the combination inhibits proliferation of cancer cells.

In another embodiment is a method for reducing proliferation of cancer cells, the method comprising delivering to the cells a combination comprising a 1,2-diphenylpyrrole derivative and a microtubule inhibitor, whereby the reduction of cell proliferation is greater than a reduction caused by either a 1,2-diphenylpyrrole derivative alone or a microtubule inhibitor alone.

In another embodiment is a method of inhibiting metastases of tumor cells, the method comprising administering an effective amount of a combination comprising a 1,2-diphenylpyrrole derivative and a microtubule inhibitor such that the combination inhibits metastatic activity of tumor cells.

In another embodiment is a method for inducing apoptosis in cancer cells, the method comprising contacting the cancer cells with a combination comprising a 1,2-diphenylpyrrole derivative and a microtubule inhibitor sufficient to induce apoptosis.

In another embodiment is a method for sensitizing cancer cells to the presence of a microtubule inhibitor wherein said cancer cells have developed resistance to treatment with a microtubule inhibitor, the method comprising administering a combination comprising a 1,2-diphenylpyrrole derivative and a microtubule inhibitor wherein the combination sensitizes the cancer cells to the microtubule inhibitor.

In another embodiment is a method of treating resistance in a cancer cell to treatment with a microtubule inhibitor the method comprising, administering a combination comprising a 1,2-diphenylpyrrole derivative and a microtubule inhibitor.

In another embodiment is a method of modulating prostaglandin synthesis in a cancer cell, the method comprising contacting the cell with a combination comprising a 1,2-diphenylpyrrole derivative and a microtubule inhibitor wherein the combination inhibits prostaglandin synthesis in a cancer cell.

In another embodiment is a method of modulating cyclooxygenase expression in a cancer cell, the method comprising delivering to the cell a combination comprising a 1,2-diphenylpyrrole derivative and a microtubule inhibitor wherein the combination inhibits cyclooxygenase expression in a cancer cell.

In another embodiment is a method of modulating angiogenesis in a cancer cell, the method comprising contacting the cell with a combination comprising a 1,2-diphenylpyrrole derivative and a microtubule inhibitor wherein the combination inhibits angiogenesis in a cancer cell.

In another embodiment is a method of reducing the dosage in conventional treatment for neoplasia and/or neoplasia-related disorders in a subject, the method comprising administering to a subject a combination of a 1,2-diphenylpyrrole derivative and a microtubule inhibitor wherein the combination reduces the dosage in conventional treatment for neoplasia and/or neoplasia-related disorders.

In another embodiment is a method of treating neoplasia and/or neoplasia-related disorders, the method comprising administering a combination of a 1,2-diphenylpyrrole derivative and a microtubule inhibitor.

In another embodiment is a combination therapy for treating cancer comprising, a combination of a 1,2-diphenylpyrrole derivative and a microtubule inhibitor or their respective pharmaceutically acceptable salt, solvate or prodrug.

In another embodiment is the combination therapy wherein the 1,2-diphenylpyrrole derivative has the following formula:

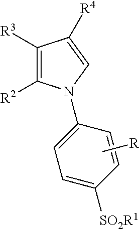

wherein:
R is a hydrogen atom, a halogen atom or an alkyl group having from 1 to 6 carbon atoms;
R¹ is an alkyl group having from 1 to 6 carbon atoms or an amino group;
R² is a phenyl group which is unsubstituted or is substituted by at least one substituent selected from the group consisting of substituents α and substituents β;
R³ is a hydrogen atom, a halogen atom or an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms;
R⁴ is a hydrogen atom; an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms; a cycloalkyl group having from 3 to 8 carbon atoms, an aryl group; or an aralkyl group; said aryl group having from 6 to 14 ring carbon atoms in a carbocyclic ring and are unsubstituted or are substituted by at least one substituent selected from the group consisting of substituents α and substituents β;
said aralkyl group are an alkyl group having from 1 to 6 carbon atoms and which are substituted by at least one aryl group as defined above;
said substituents α are selected from the group consisting of a hydroxy group, a halogen atom, an alkoxy group having from 1 to 6 carbon atoms and an alkylthio group having from 1 to 6 carbon atoms;
said substituents β are selected from the group consisting of an alkyl group which has from 1 to 6 carbon atoms and which is unsubstituted or are substituted by at least one substituent selected from the group consisting of a hydroxy group, a halogen atom, an alkoxy group having from 1 to 6 carbon atoms and an alkylthio group having from 1 to 6 carbon atoms; an alkanoyloxy group having from 1 to 6 carbon atoms; a mercapto group; an alkanoylthio group having from 1 to 6 carbon atoms; an alkylsulfinyl group having from 1 to 6 carbon atoms; a cycloalkloxy group having from 3 to 8 carbon atoms; a haloalkoxy group having from 1 to 6 carbon atoms; and an alkylenedioxy group having from 1 to 6 carbon atoms; or a pharmaceutically acceptable salt, solvate, or prodrug.

In another embodiment is the combination therapy wherein the 1,2-diphenylpyrrole derivative has the following formula:

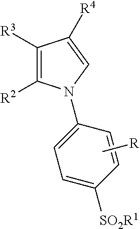

wherein:
R is a hydrogen atom, a halogen atom or an alkyl group having from 1 to 4 carbon atoms;
R¹ is a methyl group or an amino group;
R² is an unsubstituted phenyl group or a phenyl group which is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and which is substituted by at least one substituent selected from the group consisting of a halogen atom, an alkoxy group having from 1 to 4 carbon atoms and an alkylthio group having from 1 to 4 carbon atoms; a haloalkoxy group having from 1 to 4 carbon atoms; and an allylenedioxy group having from 1 to 4 carbon atoms;
R³ is a hydrogen atom, a halogen atom, an unsubstituted alkyl group having from 1 to 4 carbon atoms or a substituted alkyl group having from 1 to 4 carbon atoms and substituted by at least one substituent selected from the group consisting of a halogen atom, an alkoxy group having from 1 to 4 carbon atoms and an alkylthio group having from 1 to 4 carbon atoms;
R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to carbon atoms; a cycloalkyl group having from 3 to 6 carbon atoms; an aryl group which has from 6 to 10 ring carbon atoms and which is unsubstituted or is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to 4 carbon atoms; and a cycloalkyloxy group having from 3 to 7 carbon atoms; an aralkyl group having from 1 to 4 carbon atoms in the alkyl part and containing at least one said aryl group; or a pharmaceutically acceptable salt, solvate, or prodrug.

In another embodiment is the combination therapy wherein the 1,2-diphenylpyrrole derivative has the following formula:

[symbol]

In another embodiment is the combination therapy wherein the microtubule inhibitor is docetaxel.

In another embodiment is the combination therapy wherein the 1,2-diphenylpyrrole derivative is 2-(4-ethoxyphenyl)-4-methyl-1-(4-sulfamoylphenyl)-pyrrole and the microtubule inhibitor is docetaxel.

In another embodiment is the combination therapy wherein the 1,2-diphenylpyrrole derivative is in an oral, parenteral, buccal, intranasal, epidural, sublingual, pulmonary, local, rectal, or transdermal form.

In another embodiment is the combination therapy wherein the 1,2-diphenylpyrrole derivative is in the parenteral form.

In another embodiment is the combination therapy wherein the parenteral form is intravenous, subcutaneous, intrathecal, or intramuscular.

In another embodiment is the combination therapy wherein the combination therapy is suitable for once-daily administration.

In another embodiment is the combination therapy wherein the combination therapy contains a lower dose than a conventional treatment for cancer.

In another embodiment is the combination therapy wherein the combination therapy reduces the side effects of the treatment of cancer.

In another embodiment is the combination therapy wherein the combination therapy enhances treatment of cancer.

In another embodiment is the combination therapy for treating cancer comprising, a combination of a 1,2-diphenylpyrrole derivative and a microtubule inhibitor, and a pharmaceutically acceptable excipient or carrier.

In another embodiment is the combination therapy wherein the 1,2-diphenylpyrrole derivative is 2-(4-ethoxyphenyl)-4-methyl-1-(4-sulfamoylphenyl)-pyrrole.

In another embodiment is the combination therapy wherein the microtubule inhibitor is docetaxel.

In another embodiment is the combination therapy wherein the 1,2-diphenylpyrrole derivative is 2-(4-ethoxyphenyl)-4-methyl-1-(4-sulfamoylphenyl)-pyrrole and the microtubule inhibitor is docetaxel.

In another embodiment is the method wherein the cancer to be treated is breast cancer.

In another embodiment is the method wherein the cancer to be treated is breast cancer.

In another embodiment is the method wherein the cancer to be treated is breast cancer.

In another embodiment is the method wherein the cancer to be treated is breast cancer.

In another embodiment is the method wherein the cancer to be treated is non-small cell lung cancer.

In another embodiment is the method wherein the cancer to be treated is non-small cell lung cancer.

In another embodiment is the method wherein the cancer to be treated is non-small cell lung cancer.

In another embodiment is the method wherein the cancer to be treated is non-small cell lung cancer.

In another embodiment is the method comprising administering to the patient 2-(4-ethoxyphenyl)-4-methyl-1-(4-sulfamoylphenyl)-pyrrole at a dose of about 400 mg/day and docetaxel at a dose of 75 mg/m² administered intravenously on day 1 of every 21-day cycle.

In another embodiment is the method comprising administering to the patient 2-(4-ethoxyphenyl)-4-methyl-1-(4-sulfamoylphenyl)-pyrrole at a dose of from about 100 to about 1200 mg/day and docetaxel at a dose of about 25 to about 150 mg/m² administered intravenously on day 1 of each 21-day cycle.

In another embodiment is the method further comprising administering cisplatin or carboplatin as an additional therapy.

In another embodiment is the method further comprising administering to the subject one or more therapies in addition to the combination of a 1,2-diphenylpyrrole derivative and a microtubule inhibitor.

In another embodiment is the method wherein the cancer to be treated is breast cancer.

In another embodiment is the method wherein the cancer to be treated is non-small cell lung cancer.

In another embodiment is the method wherein the one or more therapies comprise one or more of radiation therapy, chemotherapy, high dose chemotherapy with stem cell transplant, hormone therapy, and monoclonal antibody therapy.

In another embodiment is the method wherein radiation therapy comprises internal and/or external radiation therapy.

In another embodiment is the method wherein the chemotherapy comprisies administering to the subject one or more of erlotinib, bendamustine, carboplatin, carmustine, chlorambucil, cisplatin, cyclophosphamide injection, cyclophosphamide, dacarbazine, ifosfamide, lomustine, mechlorethamine, melphalan, procarbazine, bleomycin, doxorubicin, epirubicin, idarubicin, mitoxantrone, gemcitabine, mercaptopurine, pentostatin IV, thioguanine, etoposide, etoposide dexamethasone, methylprednisolone, or prednisone.

In another embodiment is the method further comprising administering to the subject one or more therapies in addition to the combination of 2-(4-ethoxyphenyl)-4-methyl-1-(4-sulfamoylphenyl)-pyrrole and docetaxel.

In another embodiment is the method wherein the cancer to be treated is breast cancer.

In another embodiment is the method wherein the cancer to be treated is non-small cell lung cancer.

In another embodiment is the method wherein the one or more therapies comprise one or more of radiation therapy, chemotherapy, high dose chemotherapy with stem cell transplant, hormone therapy, and monoclonal antibody therapy.

In another embodiment is the method wherein radiation therapy comprises internal and/or external radiation therapy.

In another embodiment is the method wherein the chemotherapy comprisies administering to the subject one or more of erlotinib, lapatinib, capecitabine, carboplatin, carmustine, chlorambucil, cisplatin, cyclophosphamide injection, cyclophosphamide, dacarbazine, ifosfamide, lomustine, mechlorethamine, melphalan, procarbazine, bleomycin, doxorubicin, epirubicin, idarubicin, mitoxantrone, gemcitabine, mercaptopurine, pentostatin IV, thioguanine, etoposide, etoposide IV, dexamethasone, methylprednisoloneor, prednisone, and bendamustine.

In another embodiment is a method of modulating the immune response in a cancer cell, the method comprising contacting the cell with a combination comprising a 1,2-diphenylpyrrole derivative and a microtubule inhibitor wherein the combination modulates the immune response in a cancer cell.

In another embodiment is the method wherein administering to the subject the combination of 2-(4-ethoxyphenyl)-4-methyl-1-(4-sulfamoylphenyl)-pyrrole and docetaxel provides increased tumor growth delay compared to administering docetaxel alone.

In another embodiment is the method wherein administering to the subject the combination of 2-(4-ethoxyphenyl)-4-methyl-1-(4-sulfamoylphenyl)-pyrrole and docetaxel provides at least about 100% increase in tumor growth delay compared to administering docetaxel alone.

In another embodiment is the method wherein hormone therapy comprises administering to the subject tamoxifen, letrozole, anastrozole or exemestane.

In another embodiment is the method wherein hormone therapy comprises administering to the subject tamoxifen, letrozole, anastrozole or exemestane.

In another embodiment is the method wherein the 1,2-diphenylpyrrole derivative is 2-(4-ethoxyphenyl)-4-methyl-1-(4-sulfamoylphenyl)-pyrrole and the microtubule inhibitor is palcitaxel.

In another embodiment is the method wherein the 1,2-diphenylpyrrole derivative is 2-(4-ethoxyphenyl)-4-methyl-1-(4-sulfamoylphenyl)-pyrrole and the microtubule inhibitor is ixabepilone.

In another embodiment is the method comprising further administration of a monoclonal antibody that selectively binds the HER2 receptor.

In another embodiment is the method wherein the monoclonal antibody that selectively binds the HER2 receptor is trastuzumab.

In another embodiment is the method further comprising the administration of an EGFR tyrosine kinase inhibitor.

In another embodiment is the method wherein the EGFR tyrosine kinase inhibitor is erlotinib.

In another embodiment is the method wherein the microtubule inhibitor is docetaxel.

In another embodiment is the method wherein the microtubule inhibitor is paclitaxel.

In another embodiment is the method wherein the microtubule inhibitor is ixabepilone.

### INCORPORATION BY REFERENCE

All publications, patents, and patent applications described in this specification are herein incorporated by reference to the same extent as if each individual publication, patent, or patent application was specifically and individually indicated to be incorporated by reference.

### BRIEF DESCRIPTIONS OF THE DRAWINGS

FIG. 1 provides a tabular summary of the MV-522 xenograph study described in Example 13.

FIG. 2 provides a graphical summary of the MV-522 xenograph study described in Example 13.

FIG. 3 provides a tabular summary of the A549 xenograph study described in Example 13.

FIG. 4 provides a graphical summary of the MV-522 xenograph study described in Example 13.

FIG. 5 provides a tabular summary of the H460 xenograph study described in Example 13.

FIG. 6 provides a graphical summary of the MV-522 xenograph study described in Example 13.

### DETAILED DESCRIPTION

To facilitate understanding of the disclosure set forth herein, a number of terms are defined below.

### DEFINITIONS

As used herein, “abnormal cell growth,” refers to cell growth that is independent of normal regulatory mechanisms (e.g., loss of contact inhibition), including the abnormal growth of normal cells and the growth of abnormal cells. This includes, but is not limited to, the abnormal growth of: (1) tumor cells (tumors), both benign and malignant, expressing an activated Ras oncogene; (2) tumor cells, both benign and malignant, in which the Ras protein is activated as a result of oncogenic mutation in another gene; (3) benign and malignant cells of other proliferative diseases in which aberrant Ras activation occurs. Examples of such benign proliferative diseases are psoriasis, benign prostatic hypertrophy, human papilloma virus (HPV), and restenosis. Abnormal cell growth, also refers to and includes the abnormal growth of cells, both benign and malignant, resulting from activity of the enzymes farnesyl protein transferase, protein kinases, protein phosphatases, lipid kinases, lipid phosphatases, or activity or transcription factors, or intracellular or cell surface receptor proteins.

“Neoplasia” as described herein, is an abnormal, unregulated and disorganized proliferation of cell growth that is distinguished from normal cells by autonomous growth and somatic mutations. As neoplastic cells grow and divide they pass on their genetic mutations and proliferative characteristics to progeny cells. A neoplasm, or tumor, is an accumulation of neoplastic cells. In some embodiments, the neoplasm can be benign or malignant.

“Metastasis,” as used herein, refers to the dissemination of tumor cells via lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

As discussed herein, “angiogenesis” is prominent in solid tumor formation and metastasis. Angiogenic factors have been found associated with several solid tumors such as rhabdomyosarcomas, retinoblastoma, Ewing sarcoma, neuroblastoma, and osteosarcoma. A tumor cannot expand without a blood supply to provide nutrients and remove cellular wastes. Tumors in which angiogenesis is important include solid tumors, and benign tumors such as acoustic neuroma, neurofibroma, trachoma and pyogenic granulomas. Angiogenesis has been associated with blood-born tumors such as leukemias, any of various acute of chronic neoplastic diseases of the bone marrow in which unrestrained proliferation of white blood cells occurs, usually accompanied by anemia, impaired blood clotting, and enlargement of the lymph nodes, liver, and spleen. It is believed that angiogenesis plays a role in the abnormalities in the bone marrow that give rise to leukemia-like tumors. Prevention of angiogenesis could halt the growth of cancerous tumors and the resultant damage to the subject due to the presence of the tumor.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” are meant to include alleviating or abrogating a disorder, disease, or condition; or one or more of the symptoms associated with the disorder, disease, or condition; or alleviating or eradicating the cause(s) of the disorder, disease, or condition itself.

The term “therapeutically effective amount” refers to the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

### Cyclooxygenase

Cyclooxygenase (COX) is an enzyme that is responsible for the formation of important biological mediators called prostanoids, including prostaglandins, prostacyclin and thromboxane. COX converts arachidonic acid, an ω-6 essential fatty acid, to prostaglandin H₂ (PGH₂), the precursor of the series-2 prostanoids. The enzyme contains two active sites: a heme with peroxidase activity, responsible for the reduction of PGG₂ to PGH₂, and a cyclooxygenase site, where arachidonic acid is converted into the hydroperoxy endoperoxide prostaglandin G₂ (PGG₂). The reaction proceeds through a hydrogen atom abstraction from arachidonic acid by a tyrosine radical generated by the peroxidase active site, then two oxygen molecules react with the arachidonic acid radical, giving PGG₂.

COX-1 is a constitutive enzyme responsible for biosynthesis of prostaglandins in the gastric mucosa and in the kidney. COX-2 is an enzyme that is produced by an inducible gene that is responsible for biosynthesis of prostaglandins in inflammatory cells. Inflammation causes induction of COX-2, leading to release of prostanoids (prostaglandin E2), which sensitize peripheral nociceptor terminals and produce localized pain hypersensitivity, inflammation and edema.

Historically, physicians have treated inflammation-related disorders with a regimen of NSAIDs such as, for example, aspirin and ibuprofen. Undesirably, however, some NSAIDs are known to cause gastrointestinal (GI) bleeding or ulcers in patients undergoing consistent long term regimens of NSAID therapy (Henry et al., Lancet, 1991, 337, 730). A reduction of unwanted side effects of common NSAIDs was made possible by the discovery that the two cyclooxygenases involved in the transformation of arachidonic acid as the first step in the prostaglandin synthesis pathway were cyclooxygenase-1 (COX-1) and cyclooxygenase-2 (COX-2).

Many common NSAIDs are now known to be inhibitors of both COX-1 and COX-2. Accordingly, when administered in sufficiently high levels, these NSAIDs not only alleviate the inflammatory consequences of COX-2 activity, but also inhibit the beneficial gastric maintenance activities of COX-1. Research into the area of arachidonic acid metabolism has resulted in the discovery of compounds that selectively inhibit the COX-2 enzyme to a greater extent than they inhibit COX-1. These COX-2 selective inhibitors are believed to offer advantages that include the capacity to prevent or reduce inflammation while avoiding harmful side effects associated with the inhibition of Cox-1. Thus, COX-2 selective inhibitors have shown great promise for use in therapies, especially in therapies that require maintenance administration, such as for pain and inflammation control.

As described herein, the terms “cyclooxygenase-2 inhibitor” and “Cox-2 inhibitor”, which can be used interchangeably herein, denote compounds which inhibit the cyclooxygenase-2 enzyme (COX-2) regardless of the degree of inhibition of the cyclooxygenase-1 enzyme (COX-1), and include pharmaceutically acceptable racemates, enantiomers, tautomers, salts, esters and prodrugs of those compounds. Thus, for purposes of the present disclosure, a compound is considered a COX-2 inhibitor although the compound inhibits COX-2 to an equal, greater, or lesser degree than it inhibits COX-1. COX-2 inhibitors herein therefore encompass many traditional non-selective NSAIDs.

### COX-2 Selective Inhibitors

COX-2 inhibitors useful according to embodiments of the present disclosure are agents and compounds that selectively or preferentially inhibit COX-2 to a greater degree than they inhibit COX-1. Such agents and compounds are termed “COX-2 selective inhibitors” herein.

In practice, in a test for selectivity of a COX-2 selective inhibitor, the observed selectivity varies depending upon the conditions under which the test is performed and on the compound being tested. For example, selectivity of a COX-2 inhibitor can be measured as a ratio of the in vitro or in vivo IC₅₀ value for inhibition of COX-1, divided by the corresponding IC₅₀ value for inhibition of COX-2 (COX-1 IC₅₀/COX-2 IC₅₀). A COX-2 selective inhibitor herein is thus any inhibitor for which COX-1 IC₅₀/COX-2 IC₅₀ is greater than 1. In various embodiments, this ratio is greater than about 2, greater than about 5, greater than about 10, greater than about 50, or greater than about 100.

In one embodiment, the invention provides a composition, comprising a combination of a 1,2-diphenylpyrrole derivative and an EGFR inhibitor wherein the ratio of selectivity of COX-2 over COX-1 inhibition is greater than about 1. In another embodiment, the invention provides a composition comprising a combination of a 1,2-diphenylpyrrole derivative and an EGFR inhibitor wherein the ratio of selectivity of COX-2 over COX-1 inhibition is greater than about 2. In a further embodiment, the invention provides a composition comprising combination of a 1,2-diphenylpyrrole derivative and an EGFR inhibitor wherein the ratio of selectivity of COX-2 over COX-1 inhibition is greater than about 5. In another embodiment, the invention provides a composition comprising a combination of a 1,2-diphenylpyrrole derivative and an EGFR inhibitor wherein the ratio of selectivity of COX-2 over COX-1 inhibition is greater than about 7.8. In yet a further embodiment, the invention provides a composition comprising a combination of a 1,2-diphenylpyrrole derivative and an EGFR inhibitor wherein the ratio of selectivity of COX-2 over COX-1 inhibition is greater than about 10. In another embodiment, the invention provides a composition comprising a combination of a 1,2-diphenylpyrrole derivative and an EGFR inhibitor wherein the ratio of selectivity of COX-2 over COX-1 inhibition is greater than about 20. In a further embodiment, the invention provides a composition comprising a combination of a 1,2-diphenylpyrrole derivative and an EGFR inhibitor wherein the ratio of selectivity of COX-2 over COX-1 inhibition is greater than about 50. In yet a further embodiment, the invention provides a composition comprising a combination of a 1,2-diphenylpyrrole derivative and an EGFR inhibitor wherein the ratio of selectivity of COX-2 over COX-1 inhibition is greater than about 100. In one embodiment, the invention provides a composition comprising a combination of a 1,2-diphenylpyrrole derivative and an EGFR inhibitor wherein 1,2-diphenylpyrrole derivative is 2-(4-ethoxyphenyl)-4-methyl-1-(4-sulfamoylphenyl)-pyrrole. In another embodiment, the invention provides a composition comprising a combination of a 1,2-diphenylpyrrole derivative and an EGFR inhibitor wherein the EGFR inhibitor is erlotinib. In a further embodiment, the invention provides a composition comprising a combination of a 1,2-diphenylpyrrole derivative and an EGFR inhibitor wherein the 1-2-diphenylpyrrole derivative is 2-(4-ethoxyphenyl)-4-methyl-1-(4-sulfamoylphenyl)-pyrrole and the EGFR inhibitor is erlotinib and wherein the ratio of selectivity of COX-2 over COX-1 inhibition is greater than about 1, about 2, about 5, about 7.8, about 10, about 20, about 50, and about 100.

In one embodiment, the invention provides a method for treating a tumor and/or tumor related disease comprising administering a combination of a 1,2-diphenylpyrrole derivative and an EGFR inhibitor wherein the ratio of selectivity of COX-2 over COX-1 inhibition is greater than about 1. In one embodiment, the invention provides a method for treating a tumor and/or tumor related disease comprising administering a combination of a 1,2-diphenylpyrrole derivative and an EGFR inhibitor wherein the ratio of selectivity of COX-2 over COX-1 inhibition is greater than about 2. In a further embodiment, the invention provides a method for treating a tumor and/or tumor related disease comprising administering a combination of a 1,2-diphenylpyrrole derivative and an EGFR inhibitor wherein the ratio of selectivity of COX-2 over COX-1 inhibition is greater than about 5. In yet a further embodiment, the invention provides a method for treating a tumor and/or tumor related disease comprising administering a combination of a 1,2-diphenylpyrrole derivative and an EGFR inhibitor wherein the ratio of selectivity of COX-2 over COX-1 inhibition is greater than about 10. In some embodiments, the invention provides methods for treating a tumor and/or tumor related disease comprising administering a combination of a 1,2-diphenylpyrrole derivative and an EGFR inhibitor wherein the ratio of selectivity of COX-2 over COX-1 inhibition is greater than about 20, than about 50, and about 100. In other embodiments, the invention provides methods for treating a tumor and/or tumor related disease comprising administering a combination of a 1,2-diphenylpyrrole derivative and an EGFR inhibitor wherein the 1-2-diphenylpyrrole derivative is 2-(4-ethoxyphenyl)-4-methyl-1-(4-sulfamoylphenyl)-pyrrole and the EGFR inhibitor is erlotinib wherein the ratio of selectivity of COX-2 over COX-1 inhibition is greater than about 1, about 2, about 5, about 7.8, about 10, about 20, about 50, and about 100.

As used herein, the term “IC₅₀” with respect to a COX-1 or COX-2 inhibitor refers to the concentration of a compound that is required to produce 50% inhibition of activity of COX-1 or COX-2. In one embodiment, 1,2-diphenylpyrrole derivatives useful in the present disclosure can have a COX-2 IC₅₀ of less than about 3 μM. In another embodiment, the 1,2-diphenylpyrrole derivative has a COX-2 IC₅₀ of less than about 2.8 μM. In yet another embodiment, the 1,2-diphenylpyrrole derivative has a COX-2 IC₅₀ of less than about 2 μM. In some embodiments, 1,2-diphenylpyrrole derivative useful in the present disclosure can have a COX-2 IC₅₀ of less than about 1 μM, less than about 0.5 μM, or less than about 0.2 μM. 1,2-Diphenylpyrrole derivatives useful in the present disclosure can have a COX-1 IC₅₀ of greater than about 1 μM, for example greater than about 20 μM. In one embodiment, the invention provides a composition comprising a combination of a 1,2-diphenylpyrrole derivative and an EGFR inhibitor wherein the IC₅₀ is less than about 1 μM. In another embodiment, the invention provides a composition comprising a combination of a 1,2-diphenylpyrrole derivative and an EGFR inhibitor wherein the IC₅₀ is less than about 0.5 μM. In a further embodiment, the invention provides a composition comprising a combination of a 1,2-diphenylpyrrole derivative and an EGFR inhibitor wherein the IC₅₀ is less than about 0.2 μM. In one embodiment the 1,2-diphenylpyrrole derivative is 2-(4-ethoxyphenyl)-4-methyl-1-(4-sulfamoylphenyl)-pyrrole. In another embodiment, the EGFR inhibitor is erlotinib. In a further embodiment the 1,2-diphenylpyrrole derivative is 2-(4-ethoxyphenyl)-4-methyl-1-(4-sulfamoylphenyl)-pyrrole and the EGFR inhibitor is erlotinib wherein the IC₅₀ is less than about 3 μM, about 2.8 μM, about 2 μM, about 1 μM, about 0.5 μM, and about 0.2 μM.

COX-2 inhibitors exhibiting a high degree of selectivity for COX-2 over COX-1 inhibition can indicate ability to reduce incidence of common NSAID-induced side effects. In one embodiment, the invention provides a composition comprising a combination of a 1,2-diphenylpyrrole derivative and an EGFR inhibitor wherein the NSAID-induced side effects are substantially diminished. For example, NSAID-induced side effects include, but are not limited to, nausea, vomiting, diarrhea, constipation, decreased appetite, rash, dizziness, headache, drowsiness, fluid retention, edema, kidney failure, liver failure, ulcers and prolonged bleeding after surgery. In another embodiment, the invention provides a composition comprising a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib and wherein the NSAID-induced side effects are substantially diminished.

A COX-2 selective inhibitor can be used in a form of a prodrug thereof. As described herein, a “COX-2 prodrug” is a compound that can be converted into an active COX-2 selective inhibitor by metabolic or simple chemical processes within the body of the subject. One example of a prodrug for a COX-2 selective inhibitor is parecoxib, for example in a form of a salt such as parecoxib sodium, which is a therapeutically effective prodrug of the tricyclic COX-2 selective inhibitor valdecoxib.

### Overexpression of COX-2 and Cancer

The overexpression of COX-2 and also the upstream and downstream enzymes of the prostaglandin synthesis pathway have been demonstrated in multiple cancer types and some pre-neoplastic lesions. Direct interactions of prostaglandins with their receptors through autocrine or paracrine pathways to enhance cellular survival or stimulate angiogenesis have been proposed as the molecular mechanisms underlying the pro-carcinogenic functions of COX enzymes. In this respect, preclinical studies suggest that COX-2 may be involved in the molecular pathogenesis of some types of lung cancer. Most of the studies point to its involvement in non-small cell lung cancer. Survival of patients with non-small cell lung cancer expressing high levels of COX-2 is markedly reduced. Treatment of humans with the selective COX-2 inhibitor celecoxib augments the antitumor effects of chemotherapy in patients with non-small cell lung cancer.

Studies indicate that prostaglandins synthesized by cyclooxygenase play a critical role in the initiation and promotion of cancer. Aberrant COX-2 expression was first reported in colorectal carcinomas and adenomas, and has now been detected in various human cancers, including those of the breast. Moreover, COX-2 is overexpressed in neoplastic lesions of the colon, breast, lung, prostate, esophagus, pancreas, intestine, cervix, ovaries, urinary bladder and head and neck (see Table 1 below).

TABLE 1
COX-2 Expression in Tumors

| Tumor Type | % Tissue expressing COX-2 |
|---|---|
| Colorectal Cancer | 70-95 |
| Non-small Cell Lung Cancer | 70-90 |
| Gastric Cancer | 45-75 |
| Pancreatic Cancer | 40-80 |
| Glioblastoma Multiforme | 40-70 |
| Bladder Cancer | 50-60 |
| Esophageal Cancer | 50-60 |
| Breast Cancer | 40-50 |
| Ovarian Cancer | 40-60 |
| Prostate Cancer | 40-60 |

COX-2 overexpression in murine mammary glands is sufficient to cause tumor formation. In several in vitro and animal models, COX-2 inhibitors have inhibited tumor growth and metastasis.

In addition to cancers per se, COX-2 is also expressed in the angiogenic vasculature within and adjacent to hyperplastic and neoplastic lesions indicating that COX-2 plays a role in angiogenesis. In both the mouse and rat, COX-2 inhibitors markedly inhibited bFGF-induces neovascularization. The utility of COX-2 inhibitors as chemopreventive, antiangiogenic and chemotherapeutic agents is described in the literature (Koki et al., Exp. Opin., Invest. Drugs, 1999, 8(10) 1623-38).

COX-2 has been shown to regulate some embodiments of tumor-associated angiogenesis. Angiogenesis is an attractive therapeutic target because it is a multi-step process that occurs in a specific sequence, thus providing several possible targets for drug action. Angiogenesis is important in two stages of tumor metastasis. The first stage where angiogenesis stimulation is important is in the vascularization of the tumor which allows the tumor cells to enter the blood stream and to circulate throughout the body. After the tumor cells have left the primary site and have settled into the secondary, metastasis site, angiogenesis must occur before the new tumor can grow and expand. Therefore, prevention of angiogenesis could lead to the prevention of metastasis of tumors and possibly contain the neoplastic growth at the primary site. Examples of agents that interfere with several of these steps include, angiostatin, endostatin, interferon alpha and COX-2 selective inhibitors that prevent the growth of cells that form new blood vessels; and protein-based compounds that simultaneously interfere with several of these targets.

Additionally, several studies have suggested that COX-2 expression is associated with parameters of aggressive breast cancer, including large tumor size, positive axillary lymph node metastases, and HER2-positive tumor status. Studies of mammary tumors in mice and rats have indicated that moderate to high COX-2 expression is related to the genesis of mammary tumors that are sensitive to treatment with nonspecific and specific COX-2 inhibitors. Further studies have shown that increased amounts of prostaglandins and COX-2 are commonly found in a wide range of premalignant tissues and malignant tumors including cervical dysplasia and cancer. Elevated prostaglandin and COX-2 levels substantially contribute to carcinogenesis by inhibiting apoptosis and stimulating angiogenesis (Tsujii and DuBois, Cell, 1995, 83, 493-501).

Further, COX-2 is also highly expressed in prostate cancer, particularly in the epithelial cell of high-grade prostatic intraepithelial neoplasia and cancer. It was shown that treatment of human prostate cancer cell lines with a selective COX-2 inhibitor induces apoptosis both in vitro and in vivo. The in vivo results also indicate that the COX-2 inhibitor decreases tumor microvessel density and angiogenesis. COX-2 inhibitors can prevent the hypnoxic upregulation of a potent angiogenic factor, vascular endothelial growth factor.

Overexpression of COX-2 has been documented in several premalignant and malignant tissues (Subbaramaiah et al., Trends Pharmacol. Sci., 2003, 24, 96-102). Without wishing to be bound by any particular theory, this increase in expression is thought to be a product of stimulation of PKC signaling, which stimulates activity of MAPK, enhancing transcription of COX-2 by nuclear factors. Additionally, enhanced stability of COX-2 mRNA transcripts in cancer cells due to augmented binding of the RNA-binding protein HuR, as well as activation of extracellular signal related kinase 1 and 2 (ERK 1 and 2) and p38, is thought to contribute to increased expression of COX-2.

These results indicate that COX-2 inhibitors may serve as effective chemopreventive and therapeutic agents in cancer of the prostate.

### COX-2 Selective Inhibitors

It has recently been found that the use of nonsteroidal anti-inflammatory drugs (NSAIDs) has been associated with the prevention and treatment of several types of cancer (Thun et al., J. National Cancer Inst., 2002, 94(4), 252-66). COX-2 inhibitors have been utilized for the treatment of cancer and for the treatment of tumors. For example, celecoxib, a COX-2 selective inhibitor, exerted a potent inhibition of fibroblast growth factor-induced corneal angiogenesis in rats (Masferrer et al., Proc. Am. Assoc. Cancer Research, 1999, 40, 396). Other COX-2 inhibitors have been described for the treatment of cancer, tumors and neoplasia. FR 27 71 005 describes compositions containing a COX-2 inhibitor and N-methyl-d-aspartate (NMDA) antagonist used to treat cancer and other diseases. Further, WO 99/18960 describes a combination comprising a COX-2 inhibitor that can be used to treat colorectal and breast cancer. Additionally, WO 97/36497 describes a combination comprising a COX-2 inhibitor and a 5-lipoxygenase inhibitor useful in treating cancer.

### 1,2-Diphenylpyrrole Derivatives

1,2-Diphenylpyrrole derivatives and pharmaceutically acceptable salts, solvates, or prodrugs are known to have analgesic and antiphlogistic properties. Further, they have been shown to act as COX-2 selective inhibitors and are thus effective for the prophylaxis and therapy of diseases mediated by COX-2 and/or inflammatory cytokines. In addition, 1,2-diphenylpyrrole derivatives have been shown to treat diseases involving or resulting from the resorption of bone, such as osteoporosis, rheumatoid arthritis and osteoarthritis.

These types of analgesics, anti-inflammatory agents and/or antipyretics exhibit effects not only on inflammatory diseases; such as pain, pyrexia, and edema, but also on chronic inflammatory diseases, such as chronic rheumatoid arthritis and osteoarthritis, allergic inflammatory diseases, asthma, sepsis, psoriasis, various autoimmune diseases, systemic lupus erythematosus, juvenile onset diabetes, autoimmune intestinal diseases (such as ulcerative colitis, Crohn's disease), viral infection, and glomerulonephritis.

1,2-Diphenylpyrrole derivatives described herein have the general formula:

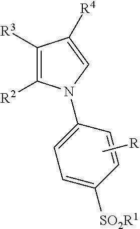

wherein:

R is a hydrogen atom, a halogen atom or an alkyl group having from 1 to 6 carbon atoms;

R¹ is an alkyl group having from 1 to 6 carbon atoms or an amino group;

R² is a phenyl group which is unsubstituted or is substituted by at least one substituent selected from the group consisting of substituents α and substituents β;

R³ is a hydrogen atom, a halogen atom or an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms;

R⁴ is a hydrogen atom; an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms; a cycloalkyl group having from 3 to 8 carbon atoms, an aryl group; or an aralkyl group; said aryl group having from 6 to 14 ring carbon atoms in a carbocyclic ring and are unsubstituted or are substituted by at least one substituent selected from the group consisting of substituents α and substituents β; said aralkyl group are an alkyl group having from 1 to 6 carbon atoms and which are substituted by at least one aryl group as defined above;

said substituents α are selected from the group consisting of a hydroxy group, a halogen atom, an alkoxy group having from 1 to 6 carbon atoms and an alkylthio group having from 1 to 6 carbon atoms;

said substituents β are selected from the group consisting of an alkyl group which has from 1 to 6 carbon atoms and which is unsubstituted or are substituted by at least one substituent selected from the group consisting of a hydroxy group, a halogen atom, an alkoxy group having from 1 to 6 carbon atoms and an alkylthio group having from 1 to 6 carbon atoms; an alkanoyloxy group having from 1 to 6 carbon atoms; a mercapto group; an alkanoylthio group having from 1 to 6 carbon atoms; an alkylsulfinyl group having from 1 to 6 carbon atoms; a cycloalkloxy group having from 3 to 8 carbon atoms; a haloalkoxy group having from 1 to 6 carbon atoms; and an alkylenedioxy group having from 1 to 6 carbon atoms; or a pharmaceutically acceptable salt, solvate, or prodrug.

In one embodiment, the invention provides a 1,2-diphenylpyrrole derivative having the formula:

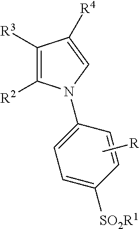

wherein:

R is a hydrogen atom, a halogen atom or an alkyl group having from 1 to 4 carbon atoms;

R¹ is a methyl group or an amino group;

R² is an unsubstituted phenyl group or a phenyl group which is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and which is substituted by at least one substituent selected from the group consisting of a halogen atom, an alkoxy group having from 1 to 4 carbon atoms and an alkylthio group having from 1 to 4 carbon atoms; a haloalkoxy group having from 1 to 4 carbon atoms; and an alkylenedioxy group having from 1 to 4 carbon atoms;

R³ is a hydrogen atom, a halogen atom, an unsubstituted alkyl group having from 1 to 4 carbon atoms or a substituted alkyl group having from 1 to 4 carbon atoms and substituted by at least one substituent selected from the group consisting of a halogen atom, an alkoxy group having from 1 to 4 carbon atoms and an alkylthio group having from 1 to 4 carbon atoms;

R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to carbon atoms; a cycloalkyl group having from 3 to 6 carbon atoms; an aryl group which has from 6 to 10 ring carbon atoms and which is unsubstituted or is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to 4 carbon atoms; and a cycloalkyloxy group having from 3 to 7 carbon atoms; an aralkyl group having from 1 to 4 carbon atoms in the alkyl part and containing at least one said aryl group; or a pharmaceutically acceptable salt, solvate, or prodrug.

In one embodiment, the invention provides a 1,2-diphenylpyrrole derivative wherein:

R is a hydrogen atom;

R¹ is an amino group;

R² is an unsubstituted phenyl group or a phenyl group which is substituted by at least one substituent selected from the group consisting of a halogen atom, an alkoxy group having from 1 to 4 carbon atoms, an alkylthio group having from 1 to 4 carbon atoms, an alkyl group having from 1 to 4 carbon atoms, a haloalkyl group having from 1 to 4 carbon atoms, a haloalkoxy group having from 1 to 4 carbon atoms and a alkylenedioxy group having from 1 to 4 carbon atoms;

R³ is a hydrogen atom, a halogen atom, an alkyl group having from 1 to 4 carbon atoms or a haloalkyl group having from 1 to 4 carbon atoms;

R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group and an alkoxy group having from 1 to 4 carbon atoms; a cycloalkyl group having from 3 to 6 carbon atoms; an aryl group which has from 6 to 10 ring carbon atoms and which is unsubstituted or is substituted by at least one substituent selected from the group consisting of a hydroxy group; a halogen atom; an alkoxy group having from 1 to 4 carbon atoms; an unsubstituted alkyl group having from 1 to 4 carbon atoms; an alkyl group having from 1 to 4 carbon atoms and which is unsubstituted or substituted by at least one halogen atom; and a cycloalkyloxy group having from 3 to 7 carbon atoms; and an aralkyl group having from 1 to 4 carbon atoms in the alkyl part and containing at least one said aryl group; or a pharmaceutically acceptable salt, solvate, or prodrug.

In one embodiment, R is a hydrogen atom. In another embodiment, R is a fluorine atom. In a further embodiment, R is a chlorine atom. In yet a further embodiment, R is a methyl group.

In one embodiment, R¹ is a methyl group. In another embodiment, R¹ is an amino group.

In one embodiment, R² is a phenyl group.

In one embodiment, R³ is a hydrogen atom. In another embodiment, R³ is a halogen atom.

In one embodiment, R⁴ is a hydrogen atom.

The term “aryl” refers to a carbocyclic aromatic hydrocarbon group having from 6 to 14 carbon atoms in one or more aromatic rings or such a group which is fused to a cycloalkyl group having from 3 to 10 carbon atoms, and the group is unsubstituted or it is substituted by at least one substituent selected from the group consisting of hydroxy groups, halogen atoms, lower alkoxy groups, lower alkylthio groups, lower alkyl groups, alkanoyloxy groups, mercapto groups, alknoylthio groups, lower alkylsulfinyl groups, lower alkyl groups having at least one substituent selected from the group consisting of cycloalkloxy groups, lower haloalkoxy groups, and lower alkylenedioxy groups.

In some embodiments, the 1,2-diphenylpyrrole derivative is selected from the group consisting of compounds 2-1-2-213 of Table 2 as disclosed in U.S. Pat. No. 6,887,893, which is herein incorporated in its entirety by reference.

In one embodiment, the 1,2-diphenylpyrrole derivative is selected from the group consisting of 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole. In another embodiment, the invention provides a method wherein the 1,2-diphenylpyrrole derivative is 2-(4-ethoxyphenyl)-4-methyl-1-(4-sulfamoylphenyl)-pyrrole.

In another embodiment, the 1,2-diphenylpyrrole derivative is 2-(4-ethoxyphenyl)-4-methyl-1-(4-sulfamoylphenyl)-pyrrole.

The methods for synthesizing 1,2-diphenylpyrrole derivatives, illustrated herein, are described in the Examples section and in U.S. RE39,420, which is incorporated herein by reference in its entirety.

### Receptor Tyrosine Kinases

Protein tyrosine kinases are a class of enzymes that catalyze the transfer of a phosphate group from ATP or GTP to the tyrosine residue located on protein substrates. Protein tyrosine kinases clearly play a role in normal cell growth. Many of the growth factor receptor proteins function as tyrosine kinases and it is by this process that they effect signaling. The interaction of growth factors with these receptors is a necessary event in normal regulation of cell growth. Under certain conditions, however, as a result of either mutation or overexpression, these receptors can become deregulated; the result of which is uncontrolled cell proliferation which can lead to tumor growth and ultimately to the disease known as cancer (Wilks, Adv. Cancer Res., 1993, 60, 43). Among the growth factor receptor kinases and their proto-oncogenes that have been identified and which are targets of the combinations presented herein are the epidermal growth factor receptor kinase (EGFR kinase, the protein product of the erbB oncogene), and the product produced by the erbB-2 (also referred to as the neu or HER2) oncogene. Since the phosphorylation event is a necessary signal for cell division to occur and since overexpressed or mutated kinases have been associated with cancer, an inhibitor of this event, a protein tyrosine inhibitor, will have therapeutic value for the treatment of cancer and other diseases characterized by uncontrolled or abnormal cell growth. For example, overexpression of the receptor kinase product of the erbB-2 oncogene has been associated with human breast and ovarian cancers (Slamon et. al., Science, 1989, 244, 707). Deregulation of EGFR kinase has been associated with epidermoid tumors and tumors involving other major organs. Because of the importance of the role played by deregulated receptor kinases in the pathogenesis of cancer, many recent studies have dealt with the development of specific PTK inhibitors as potential anti-cancer therapeutic agents.

Receptor tyrosine kinases span the cell membrane and possess an extracellular binding domain for growth factors such as epidermal growth factor (EGF), a transmembrane domain, and an intracellular portion which functions as a kinase to phosphorylate specific tyrosine kinase residues in proteins and hence to influence cell proliferation. The EGF receptor tyrosine kinase family has four members: EGFR (HER1, erbB1); HER2(c-erbB2, erbB2, neu); HER3 (erbB3); and HER4 (erbB4). The ErbB receptors generally transduce signals through two pathways. It is known that such kinases are frequently and aberrantly expressed in common human cancers such as breast cancer, gastrointestinal cancer of colon, rectum or stomach, leukemia, and ovarian, bronchial or pancreatic cancer. M discussed previously, epidermal growth factor receptor (EGFR), is mutated and/or overexpressed in many human cancers such as brain, lung, squamous cell, bladder, gastric, breast, head and neck, oesophageal, gynecological and thyroid tumors.

### Epidermal Growth Factor Receptors

Control of cell growth is regulated by the interaction of soluble growth factors and cell membrane receptors. The first step in the mitogenic stimulation of epidermal cells is the specific binding of epidermal growth factor (EGF) to a membrane glycoprotein known as the epidermal growth factor receptor (EGFR). The EGFR is composed of 1,186 amino acids which are divided into an extracellular portion of 621 residues and a cytoplasmic portion of 542 residues connected by a single hydrophobic transmembrane segment of 23 residues (Ullrich et al., Nature, 1986, vol. 309, 418-25). The external portion of the EGFR can be subdivided into four domains. Recently, it has been demonstrated that domain III, residues 333 to 460 which is flanked by two cysteine domains is likely to contain the EGFR binding site of the receptor (Lax et al., Mol. And. Cell Biol., 1988, vol. 8, 1831-34). The binding of EGF to domain III leads to the initiation of pleiotropic responses leading to DNA synthesis and cell proliferation.

EGFR has also been found in various types of human tumor cells and that those cells overexpress EGFR. For example, the cancerous cells of bladder tumors have been shown to have a relatively large population of EGF receptors (Neal et al., Lancet, 1985, vol. 1, 366-67). Breast cancer cells exhibit a positive correlation between EGFR density and tumor size and a negative correlation with the extent of differentiation (Sainsbury et al., Lancet, 1985, vol. 1, 364-66). The tumorigenicity of a series of human vulval epidermoid carcinoma (A431) clonal variants implanted into athymic mice having different levels of EGFR was found to correlate directly with the level of expression of the EGF receptor (Santon et al., Cancer Res., 1986, vol. 46, 4700-01). Thus, it has been proposed that overexpression of EGFRs play a role in the origin or tumorigenesis of cancer cells.

### EGFR and COX Pathways

The relationship between the EGFR and COX pathways, to date, has not yet been fully elucidated. It has been submitted, however, that induction of COX-2 results in the production of increased levels of prostaglandins which then stimulate angiogenesis, cell proliferation and cell differentiation in an autocrine and/or paracrine manner. As described above, NSAIDs inhibit this process. Prostaglandins promote angiogenesis, along with cellular differention and proliferation. They also activate signaling through the EGFR. EGFR activation leads to phosphorylation on tyrosine residues by the receptor's tyrosine kinase domain, initiating a signaling pathway that includes the molecules Grb-2, SOS, the small G protein Raf. Raf activates Mitogen-Activated Protein Kinase Kinase (MAPKK) and group of nuclear transcription factors (c-myc, c-fos, c-jun). These factors initiate transcription of genes involved in the regulation of cell proliferation and differentiation. Additionally, these factors induce transcription of the COX-2. These effects may significantly amplify the original EGFR mediated signal and lead to pro-neoplastic effects Inhibiting both signaling pathways could lead to a significant anti-neoplastic effect.

Studies have provided support that EGFR induces COX-2 in intestinal epithelial cells and anti-HER2 antibodies can inhibit COX-2 expression in colorectal cancer cells. In addition, overexpression of COX-1 or COX-2 in colon carcinoma cells has been shown to increase EGFR expression. Thus, COX and EGFR levels appear to be linked in a positive feedback cycle during colon cancer development. While the exact mechanism by which dysregulated EGF signaling promotes colon carcinogenesis is not clearly understood, it has been submitted that EGFR activation in a variety of cell types results in stimulation of cell proliferation and alterations in cell motility and/or adhesion to extra-cellular matrix. Studies have suggested that COX can positively influence tumor growth by promoting tumor associated angiogenesis.

It has also recently been proposed that the activation and overexpression of COX-2 in adenomatous polyps is due to activation of the EGFR. EGFR stimulation by one of its ligands, amphiregulin (AR), induces the nuclear targeting of COX-2, release of prostaglandins and subsequent mitogenesis, in polarized colonic epithelial cells. COX-2 inhibitors have been shown to prevent this series of events.

### EGFR Inhibitors

Accordingly, it has been recognized that inhibitors of receptor tyrosine kinases are useful as selective inhibitors of the growth of mammalian cancer cells. For example, erbstatin, a tyrosine inhibitor, selectively attenuates the growth in athymic nude mice of a transplanted human mammary carcinoma which expresses EGFR but is without effect on the growth of another carcinoma which does not express the EGFR.

Various other compounds, such as styrene derivatives, have also been shown to possess tyrosine inhibitory properties. Others have disclosed that certain quinazoline derivatives possess anti-cancer properties which result from their tyrosine inhibitory properties.

As described herein, an “EGFR inhibitor” is a molecule which inhibits the kinase domain of the epidermal growth factor receptor. Compounds which are EGFR inhibitors can readily be identified by one skilled in the art using methods such as, for example, standard pharmacological test procedures which measure the inhibition of the phosphorylation of the tyrosine residue of a peptide substrate catalyzed by EGFR. Briefly, the peptide substrate (RR-SRC) has the sequence arg-arg-leu-ile-glu-asp-ala-glu-tyr-ala-ala-arg-gly. The enzyme is obtained as a membrane extract of A431 cells (American Type Culture Collection, Rockville, Md.). A431 cells are grown in T175 flasks to 80% confluency. The cells are washed twice with phosphate buffered saline (PBS) without Ca²⁺. Flasks are rotated for 1.5 hours in 20 ml PBS with 1.0 mM ethylenediaminetetraacetic acid (EDTA) at room temperature and centrifuged at 600 g for 10 minutes. The cells are solubilized in 1 ml per 5×10⁶ cells of cold lysis buffer {10 mM 4-(2-hydroxyethyl)-1-piperazineethanesulfonic acid (HEPES), pH 7.6, 10 mM NaCl, 2 mM EDTA, 1 mM phenylmethylsulfonyl-fluoride (PMSF), 10 mg/ml aprotinin, 10 mg/ml leupeptin, 0.1 mM sodium orthovanadate) in a Dounce homogenizer with 10 strokes on ice. The lysate is centrifuged at 600 g for 10 minutes first to clear cell debris and the supernatant further centrifuged at 100,000 g for 30 min at 4.degree. C. The membrane pellet is suspended in 1.5 ml HNG buffer (50 mM HEPES, pH 7.6, 125 mM NaCl, 10% glycerol). The membrane extract is divided into aliquots, immediately frozen in liquid nitrogen and stored at −70° C.

Compositions to be evaluated are made into 10 mg/ml stock solutions in 100% dimethylsulfoxide (DMSO). Stock solutions are diluted to 500 mM with buffer (30 mM Hepes pH 7.4) and then serially diluted to the desired concentration. An aliquot of the A431 membrane extract (10 mg/ml) is diluted in 30 mM HEPES (pH 7.4) to give a protein concentration of 50 μml. To 4 μl of enzyme preparation, EGF (1 μl at 12 μg/ml) is added and incubated for 10 min on ice followed by 4 μl of the test compound or buffer; this mix is incubated on ice for 30 min. To this is added the ³³P-ATP (10 mCi/ml) diluted 1:10 in assay buffer along with the substrate peptide at a concentration of 0.5 mM (control reactions get no test compound) and the reaction is allowed to proceed for 30 min at 30° C. The reaction is stopped with 10% TCA and left on ice for at least 10 min after which tubes are microcentrifuged at full speed for 15 min. A portion of the supernatants are then spotted on P81 phosphocellulose discs and washed twice in 1% acetic acid then water for 5 min each followed by scintillation counting. The results obtained can be expressed as an IC₅₀. The IC₅₀ is the concentration of test compound needed to reduce the total amount of phosphorylated substrate by 50%. The % inhibition of the test compound is determined for at least three different concentrations and the IC₅₀ value is evaluated from the dose response curve. The % inhibition is evaluated with the following formula:

% inhibition=100−[CPM(drug)/CPM(control)]×100

where CPM(drug) is in units of counts per minute and is a number expressing the amount of radiolabeled ATP (g-³³P) incorporated onto the RR-SRC peptide substrate by the enzyme after 30 minutes at 30° C. in the presence of test compound as measured by liquid scintillation counting. CPM(control) is in units of counts per minute and is a number expressing the amount of radiolabeled ATP (g-³³P) incorporated into the RR-SRC peptide substrate by the enzyme after 30 minutes at 30° C. in the absence of test compound as measured by liquid scintillation counting. The CPM values are corrected for the background counts produced by ATP in the absence of the enzymatic reaction. Compounds having an IC₅₀ of about 200 nM or less are considered to be significantly active EGFR inhibitors.

The identification of EGFR as an oncogene has led to the development of anticancer therapeutics directed against EGFR, including gefitinib and cetuximab for colon cancer. Cetuximab is an example of a monoclonal antibody inhibitor, while gefitinib is a small molecule inhibitor.

### Monoclonal Antibodies

The monoclonal antibodies block the extracellular ligand binding domain. With the binding site blocked, signal molecules can no longer attach there and activate the tyrosine kinase.

Cetuximab is a chimeric monoclonal antibody generated from fusion of the variable region of the murine anti-EGFR monoclonal antibody M225 and the human IgG1 constant region. The resulting antibody retains high affinity and specificity to EGFR and reduces immunogenicity. Preclinical studies have demonstrated that cetuximab effectively inhibits the proliferation of a variety of EGFR-expressing cancer cells in vitro and that it inhibits tumor growth in xenograft models. In an orthotopic pancreatic cancer model, cetuximab significantly suppressed the growth of orthotopically implanted pancreatic tumors, and this effect was enhanced by the addition of gemcitabine. Histologic analysis of tumor specimens revealed that cetuximab induced apoptosis and suppressed proliferation of tumor cells. Interestingly, cetuximab also induced apoptosis of endothelial cells, which are not believed to be direct targets of EGFR inhibition. Moreover, an antiangiogenic effect, characterized by decreased microvascular densities associated with reduced expression of tumor-related VEGF and interleukin-8, was observed. These data suggest that, in addition to direct antiproliferative activity, antiangiogenic activity contributes significantly to the antitumor effect of EGFR inhibitors.

Panitumumab (ABX-EGF) is a fully human monoclonal antibody specific to EGFR and is produced by immunization of transgenic mice, that are able to produce human immunoglobulin light and heavy chains. Following immunization, a specific clone of B cells that produced the antibody against EGFR are selected and immortalized for the generation of the antibody.

Effective anti-EGFR monoclonal antibodies compete with endogenous ligands, primarily EGF and transforming growth factor a for receptor ligand-binding sites. Binding to EGFR blocks critical signaling pathways and interferes with the growth of tumors expressing EGFR. Anti-EGFR monoclonal antibodies that are currently under study include EMD 55900 and ICR 62.

### Small Molecules

Another method is using small molecules to inhibit the EGFR tyrosine kinase, which is on the cytoplasmic side of the receptor. Without kinase activity, EGFR is unable to activate itself, which is a prerequisite for binding of downstream adaptor proteins. Ostensibly by halting the signaling cascade in cells that rely on this pathway for growth, tumor proliferation and migration is diminished.

Gefitinib is currently only indicated for the treatment of locally advanced or metastatic non-small cell lung cancer (NSCLC) in patients who have previously received chemotherapy. While gefitinib has yet to be proven to be effective in other cancers, there is potential for its use in the treatment of other cancers where EGFR overexpression is involved.

### Erlotinib

N-(3-ethynylphenyl)-6,7-bis(2-methoxyethoxy)quinazolin-4-amine, also known as erlotinib (Tarceva®), is currently being used to help treat second-line advanced non-small cell lung cancer and in combination with gemcitabine in the treatment for pancreatic cancer. U.S. Pat. No. 5,747,498 describes the preparation of erlotinib and other chemically-related compounds. Additionally, U.S. Pat. No. 6,900,221 describes the use of a stable polymorph of erlotinib as an inhibitor of the erbB family of oncogenic and protoncogenic protein kinases such as EGFR. Also, the patent illustrates methods for the treatment of non-small cell lung cancer, pediatric malignancies, cervical and other tumors caused or promoted by human papilloma virus (HPV), melanoma, Barrett's esophagus (pre-malignant syndrome), adrenal and skin cancers and auto immune, neoplastic cutaneous diseases and atherosclerosis.

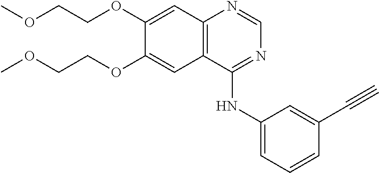

### N-(3-ethynylphenyl)-6,7-bis(2-methoxyethoxy)quinazolin-4-amine

In one embodiment the invention provides a composition comprising a combination of a COX-2 selective inhibitor and an EGFR inhibitor disclosed herein for the treatment and prevention of cancer, tumors, and tumor-related disorders, and neoplastic disease states. In one embodiment, the EGFR inhibitor is a small molecule compound or an antibody.

In one embodiment, the EGFR inhibitor is selected from gefitinib, cetuximab, and erlotinib. In another embodiment, the EGFR inhibitor is erlotinib.

In another embodiment the small molecule compound is selected from the group consisting of: ZM-254530, BIBX-1382, reveromycin A, gefitinib, CGP-57148, CGP-59326, 4-(3-chloro)-5,6-dimethyl-7H-pyrrolo[2,3-d]pyrimidine, tyrphostin, PKI-166, PD 153035, EKB-569, and 4-(phenylamino)quinazolines, or their pharmaceutically acceptable salts, solvates, or prodrugs. In another embodiment, the small molecule is selected from the compounds disclosed in PCT/US04/027574 which is herein incorporated by reference in its entirety.

In another embodiment, the antibody is selected from the group consisting of EGF receptor antibody, MR1scFvPE38KDEL MDX-447, MDX-210, MD-72000, MDX-260, wayne anti-EGFR Mabs, anti-EGFr Mab, anti-EGFr MAb, Genen anti-EGFR Mab, MAb DC-101, trastuzumab, anti-VEGF monoclonal, anti-EGFR-DM1 Ab, MAb 4D5, BAB-447, EMD-55900, EMD-6200, -82633, anti-EGFR Mab, MAb 4D5, cetuximab, anti-EGFr MAb, anti-flk-1, CCX, CCZ, anti-flk-1, AG-514, AG-568, nti-EGFR-DM1 Ab, MDX-447, TgDCC-E1A, of: muellerian-inhibiting hormone, TNP-470, tecogalan sodium, C C225, matuzumab, panitumumab, DWP-408, and RC-3940II. In another embodiment are provided compositions wherein the EGFR inhibitor is selected from the inhibitors disclosed in PCT/US04/027574 which is herein incorporated by reference in its entirety.

In a further embodiment the EGFR inhibitor is selected from the group consisting EGF receptor antisense, PI-88, oligonucleotide, bromelain molecules, amphiregulin, EGF fusion toxin, EGF fusion protein, Amphiregulin hbEGF-toxin, hbEGF-toxin, and EGF fusion protein.

As described herein “a selective EGFR inhibitory effect” is meant that the composition comprising a combination of a 1,2-diphenylpyrrole derivative with erlotinib displays selective inhibition against EGFR than other kinases. In some embodiments, combinations presently disclosed display selective inhibition against EGFR kinase than against other tyrosine kinases such as other erbBRs such as erbB2. For example in one embodiment, the invention provides a composition comprising a combination of a 1,2-diphenylpyrrole derivative with erlotinib is at least 5 times or at least 10 times selective against EGFR than it is against erbB2, as determined from the relative IC₅₀ values in suitable assays (for example by comparing the IC₅₀ value from the KB cell assay with the IC₅₀ value from the Clone 24 phospho-erbB2 cell assay for a given composition as described above). In another embodiment, the invention provides a composition comprising a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib wherein the combination is at least 5 times or at least 10 times selective against EGFR than it is against erbB2.

### Tubulin Inhibitors

### Docetaxel

Docetaxel has the chemical structure shown below. It is a highly lipophilic and practically insoluble in water. Docetaxel is provided as a concentrated viscous solution in polysorbate 80 and is diluted in an ethanol-water solution prior to administration.

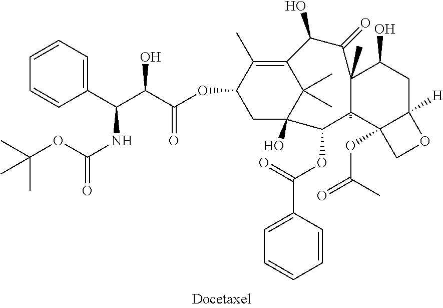

Docetaxel exerts is action by promoting the assembly of tubulin into stable microtubules while simultaneously preventing their disassembly. These stable microtubules lack normal function and inhibit cell division.

Docetaxel is indicated for the treatment of several cancers.

For breast cancer, docetaxel is indicated for the treatment of patients with locally advanced or metastatic breast cancer after failure of prior chemotherapy. In this therapy, the recommended dose of docetaxel is 60-100 mg/m² administered intravenously over 1 hour every 3 weeks. Docetaxel is also indicated for the treatment of breast cancer in combination with doxorubicin and cyclophosphamide for adjuvant treatment of patients with operable node-positive breast cancer. In this therapy, the recommended dose of docetaxel is 75 mg/m² administered 1 hour after doxorubicin (50 mg/m2) and cyclophosphamide (500 mg/m²) every 3 weeks for 6 courses.

For non-small cell lung cancer, docetaxel as a single agent is indicated for the treatment of patients with locally advanced or metastatic non-small cell lung cancer after failure of prior platinum-based chemotherapy. In this therapy, the recommended dose of docetaxel is 75 mg/m² administered intravenously over 1 hour every 3 weeks. Docetaxel is also indicated for the treatment of non-small cell lung cancer in combination with cisplatin for the treatment of patients with unresectable, locally advanced or metastatic non-small cell lung cancer who have not previously received chemotherapy. In this therapy, the recommended dose of docetaxel is 75 mg/m² administered intravenously over 1 hour immediately followed by cisplatin 75 mg/m² over 30-60 minutes every 3 weeks.

For prostate cancer, docetaxel, in combination with prednisone, is indicated for the treatment of patients with androgen independent (hormone refractory) metastatic prostate cancer. In this therapy, the recommended dose of docetaxel is 75 mg/m² administered intravenously over 1 hour every 3 weeks along with twice daily oral prednisone (5 mg).

For gastric adenocarcinoma, docetaxel, in combination with cisplatin and fluorouracil, is indicated for the treatment of patients with advanced gastric adenocarcinoma, including adenocarcinoma of the gastroesophageal junction, who have not received prior chemotherapy for advanced disease. In this therapy, the recommended dose of docetaxel is 75 mg/m² administered intravenously over 1 hour followed by cisplatin 75 mg/m² administered intravenously over 1 to 3 hours followed by fluorouracil 750 mg/m² daily administered as a 24-hour continuous intravenous infusion for 5 days, starting at the end of cisplatin infusion. Treatment is repeated every three weeks.

For head and neck cancer, docetaxel, in combination with cisplatin and fluorouracil, is indicated for the induction treatment of patients with locally advanced squamous cell carcinoma of the head and neck. In this therapy, the recommended dose of docetaxel is 75 mg/m² administered intravenously over 1 hour followed by cisplatin 75 mg/m² administered intravenously over 1 hours followed by fluorouracil 750 mg/m² daily administered as a 24-hour continuous intravenous infusion for 5 days, starting at the end of cisplatin infusion. Treatment is repeated every three weeks.

### Paclitaxel

Paclitaxel has the chemical structure shown below and is highly insoluble in water. It is supplied as a solution in polyoxyethylated castor oil and ethanol and is diluted prior to intravenous infusion.

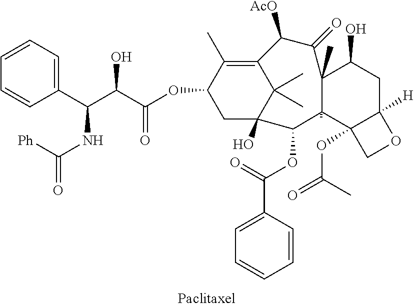

Paclitaxel exerts its action by promoting the assembly of tubulin into stable microtubules while simultaneously preventing their disassembly. These stable microtubules lack normal function and inhibit cell division.

Paclitaxel is indicated for the treatment of several cancers.

For ovarian cancer, paclitaxel is indicated for initial and follow-on therapy for the treatment of advanced carcinoma of the ovary. For initial therapy, paclitaxel is indicated in combination with cisplatin. In this therapy, the recommended dose of paclitaxel is 175 mg/m² administered intravenously over 3 hours followed by cisplatin at a dose of 75 mg/m² every 3 weeks.

For breast cancer, paclitaxel is indicated for the adjuvant treatment of node-positive breast cancer administered sequentially to standard doxorubicin-containing combination chemotherapy. Paclitaxel is also indicated for the treatment of breast cancer after failure of combination chemotherapy for metastatic disease or relapse within six months of adjuvant chemotherapy. In this therapy, the recommended dose of paclitaxel is 175 mg/m² administered intravenously over 3 hours every 3 weeks for 4 courses.

For lung cancer, paclitaxel, in combination with cisplatin, is indicated for the initial treatment of non-small cell lung cancer in patients with who are not candidates for curative surgery or radiation therapy. In this therapy, the recommended dose of paclitaxel is 135 mg/m² administered intravenously over 24 hours followed by cosplatin 75 mg/m².

Paclitaxel is indicated for the second-line treatment of AIDS-related Kaposi's sarcoma. In this therapy, the recommended dose of paclitaxel is 135 mg/m² administered intravenously over 3 hours every 3 weeks.

### Ixabepilone

Ixabepilone has the chemical structure shown below. It is supplied as a powder and administered as a solution in polyoxyethylated castor oil and ethanol.

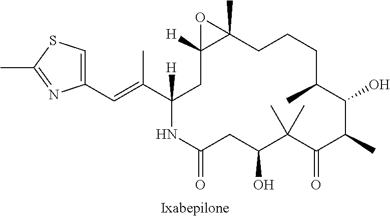

Ixabepilone exerts its action by promoting the assembly of tubulin into stable microtubules while simultaneously preventing their disassembly. These stable microtubules lack normal function and inhibit cell division.

Ixabepilone is indicated, in combination with capecitabine, for the treatment of patients with metastatic or locally advanced breast cancer resistant to treatment with an anthracycline and a taxane, or whose cancer is taxane resistant and for whom further anthracycline therapy is contraindicated. Ixabepilone is also indicated as monotherapy for the treatment of metastatic or locally advanced breast cancer in patients whose tumors are resistant or refractory to anthracyclines, taxanes and capecitabine. The recommended dosage of ixabepilone is 40 mg/m² administered intravenously over 3 hours every 3 weeks.

The compositions provided herein may be enantiomerically pure, such as a single enantiomer or a single diastereomer, or be stereoisomeric mixtures, such as a mixture of enantiomers, a racemic mixture, or a diastereomeric mixture, or a polymorph of the active agent, for example for erlotinib, polymorphs, including but not limited to polymorphs A, B, and E, and amorphous forms or solid amorphous dispersions as disclosed in US20060154941. As such, one of skill in the art will recognize that administration of a compound in its (R) form is equivalent, for compounds that undergo epimerization in vivo, to'administration of the compound in its (S) form. Conventional techniques for the preparation/isolation of individual enantiomers include chiral synthesis from a suitable optically pure precursor or resolution of the racemate using, for example, chiral chromatography, recrystallization, resolution, diastereomeric salt formation, or derivatization into diastereomeric adducts followed by separation.

When the composition described herein contains an acidic or basic moiety, it may also be provided as a pharmaceutically acceptable salt (See, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The composition described herein may also be provided as a prodrug, which is a functional derivative of the 1,2-diphenylpyrrole derivative and/or the EGFR inhibitor and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Mad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

### Methods of Use

The invention provides a method for treating a subject having tumors, tumor-related disorders, and/or cancer, comprising administering to the subject, a therapeutically effective amount of a combination comprising 2-(4-ethoxyphenyl)-4-methyl-1-(4-sulfamoylphenyl)-pyrrole and erlotinib.

In one embodiment, the invention provides a method for treating a subject having tumors, tumor-related disorders, and/or cancer, comprising administering to the subject, a therapeutically effective amount of a combination comprising a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole.

In another embodiment, the invention provides a method for treating a subject having tumors, tumor-related disorders, and/or cancer, comprising administering to the subject, a therapeutically effective amount of a combination comprising a 1,2-diphenylpyrrole derivative and an EGFR inhibitor wherein the EGFR inhibitor is erlotinib.

In yet another embodiment, the invention provides a method for treating a subject having a tumors, tumor-related disorders, and/or cancer, comprising administering to the subject, a therapeutically effective amount of a combination comprising a 1,2-diphenylpyrrole derivative and an EGFR inhibitor or their respective pharmaceutically acceptable salt, solvate or prodrug.

In one embodiment, the invention provides a method for treating a subject having tumors, tumor-related disorders, and/or cancer, comprising administering to the subject, a therapeutically effective amount of a combination comprising a 1,2-diphenylpyrrole derivative and an EGFR inhibitor wherein the 1,2-diphenylpyrrole derivative is selected from the group consisting of 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole.

In yet another embodiment, the invention provides a method for treating a subject having tumors, tumor-related disorders, and/or cancer, comprising administering to the subject, a therapeutically effective amount of a combination comprising a 1,2-diphenylpyrrole derivative and an EGFR inhibitor wherein the 1,2-diphenylpyrrole derivative and the EGFR inhibitor are administered sequentially in either order or simultaneously. In a further embodiment, the invention provides a method for treating a subject having tumors, tumor-related disorders, and/or cancer, comprising administering to the subject, a therapeutically effective amount of a combination comprising a 1,2-diphenylpyrrole derivative and an EGFR inhibitor wherein the 1,2-diphenylpyrrole derivative is administered first. In one embodiment, the invention provides a method for treating a subject having tumors, tumor-related disorders, and/or cancer, comprising administering to the subject, a therapeutically effective amount of a combination comprising a 1,2-diphenylpyrrole derivative and an EGFR inhibitor wherein the EGFR inhibitor is administered first. In another embodiment, the invention provides a method for treating a subject having tumors, tumor-related disorders, and/or cancer, comprising administering to the subject, a therapeutically effective amount of a combination comprising a 1,2-diphenylpyrrole derivative and an EGFR inhibitor wherein administering the combination enhances treatment of the subject in comparison to a treatment of either a 1,2-diphenylpyrrole derivative or an EGFR inhibitor alone. In yet another embodiment, the invention provides a method for treating a subject having tumors, tumor-related disorders, and/or cancer, comprising administering to the subject, a therapeutically effective amount of a combination comprising a 1,2-diphenylpyrrole derivative and an EGFR inhibitor wherein administering the combination reduces the side effects of the treatment of tumors, tumor-related disorders, and/or cancer.

In one embodiment, the invention provides a method for treating a subject having tumors, tumor-related disorders, and/or cancer, comprising administering to the subject, a therapeutically effective amount of a combination comprising a 1,2-diphenylpyrrole derivative and an EGFR inhibitor wherein the cancer is selected from the group consisting of oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

In another embodiment, the invention provides a method for treating a subject having tumors, tumor-related disorders, and/or cancer, comprising administering to the subject, a therapeutically effective amount of a combination comprising a 1,2-diphenylpyrrole derivative and an EGFR inhibitor wherein the cancer is non-small cell lung cancer, pancreatic cancer, breast cancer, ovarian cancer, colorectal cancer, and head and neck cancer.

In one embodiment, the invention provides a method for treating a subject having tumors, tumor-related disorders, and/or cancer, comprising administering to the subject, a therapeutically effective amount of a combination comprising a 1,2-diphenylpyrrole derivative and an EGFR inhibitor wherein the cancer is a carcinoma, a tumor, a neoplasm, a lymphoma, a melanoma, a glioma, a sarcoma, and a blastoma. In another embodiment, the invention provides a method for treating a subject having a carcinoma, comprising administering to the subject, a therapeutically effective amount of a combination comprising a 1,2-diphenylpyrrole derivative and an EGFR inhibitor wherein the carcinoma is selected from the group consisting of carcinoma, adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiatied carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial, metastatic carcinoma, mucoepidermoid carcinoma, cholangiocarcinoma, actinic keratoses, cystadenoma, and hepatic adenomatosis. In a further embodiment the tumor is selected from the group consisting of: astrocytic tumors, malignant mesothelial tumors, ovarian germ cell tumor, supratentorial primitive neuroectodermal tumors, Wilm's tumor, pituitary tumors, extragonadal germ cell tumor, gastrinoma, germ cell tumors, gestational trophoblastic tumor, brain tumors, pineal and supratentorial primitive neuroectodermal tumors, pituitary tumor, somatostatin-secreting tumor, endodermal sinus tumor, carcinoids, central cerebral astrocytoma, glucagonoma, hepatic adenoma, insulinoma, medulloepithelioma, plasmacytoma, vipoma, and pheochromocytoma. In yet another embodiment, the invention provides a method for treating a subject having a neoplasm, comprising administering to the subject, a therapeutically effective amount of a combination comprising a 1,2-diphenylpyrrole derivative and an EGFR inhibitor wherein the neoplasm is selected from the group consisting of: intaepithelial neoplasia, multiple myeloma/plasma cell neoplasm, plasma cell neoplasm, interepithelial squamous cell neoplasia, endometrial hyperplasia, focal nodular hyperplasia, hemangioendothelioma, and malignant thymoma. In one embodiment the lymphoma is selected from the group consisting of nervous system lymphoma, AIDS-related lymphoma, cutaneous T-cell lymphoma, non-Hodgkin's lymphoma, lymphoma, and Waldenstrom's macroglobulinemia. In another embodiment the melanoma is selected from the group consisting of acral lentiginous melanoma, superficial spreading melanoma, uveal melanoma, lentigo maligna melanomas, melanoma, intraocular melanoma, adenocarcinoma nodular melanoma, and hemangioma.

In a further embodiment, the invention provides a method for treating a subject having a sarcoma, comprising administering to the subject, a therapeutically effective amount of a combination comprising a 1,2-diphenylpyrrole derivative and an EGFR inhibitor wherein the sarcoma is selected from the group consisting of: adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma. In one embodiment, the glioma is selected from the group consisting of: glioma, brain stem glioma, and hypothalamic and visual pathway glioma. In another embodiment, the invention provides a method for treating a subject having a blastoma, comprising administering to the subject, a therapeutically effective amount of a combination comprising a 1,2-diphenylpyrrole derivative and an EGFR inhibitor wherein the blastoma is selected from the group consisting of: pulmonary blastoma, pleuropulmonary blastoma, retinoblastoma, neuroblastoma, medulloblastoma, glioblastoma, and hemangiblastomas.

In one embodiment the EGFR inhibitor is a small molecule compound or an antibody. In another embodiment the EGFR inhibitor is a small molecule compound is selected from the group consisting of ZM-254530, BIBX-1382, reveromycin A, gefitinib, CGP-57148, CGP-59326, 4-(m-chloro)-5,6-dimethyl-7H-pyrrolo[2,3-d]pyrimidine, tyrphostin, PKI-166, PD 153035, EKB-569, and 4-(phenylamino)quinazolines, or their pharmaceutically acceptable salts, solvates, or prodrugs. In a further embodiment, the EGFR inhibitor is an antibody is selected from the group consisting of: EGF receptor antibody, MR1scFvPE38 KDEL MDX-447, MDX-210, MD-72000, MDX-260, wayne anti-EGFR Mabs, anti-EGFr Mab, anti-EGFr MAb, Genen anti-EGFR Mab, MAb DC-101, trastuzumab, anti-VEGF monoclonal, anti-EGFR-DM1 Ab, MAb 4D5, BAB-447, EMD-55900, EMD-6200, -82633, anti-EGFR Mab, MAb 4D5, cetuximab, anti-EGFr MAb, anti-flk-1, CCX, CCZ, anti-flk-1, AG-514, AG-568, nti-EGFR-DM1 Ab, MDX-447, TgDCC-E1A, C225, matuzumab, panitumumab, DWP-408, and RC-3940II. In yet a further embodiment the EGFR inhibitor is selected from the group consisting of: muellerian-inhibiting hormone, TNP-470, tecogalan sodium, EGF receptor antisense, PI-88, oligonucleotide, bromelain molecules, amphiregulin, EGF fusion toxin, EGF fusion protein, Amphiregulin hbEGF-toxin, hbEGF-toxin, and EGF fusion protein.

In one embodiment the invention provides a method of inducing differentiation of tumor cells, the method comprising contacting the cells with an effective amount of a combination comprising a 1,2-diphenylpyrrole derivative and an EGFR inhibitor whereby the combination induces differentiation of tumor cells. In one embodiment, the invention provides a method of inducing differentiation of tumor cells, the method comprising contacting the cells with an effective amount of a combination comprising a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib.

In one embodiment the invention provides a method of inhibiting proliferation of cancer cells, the method comprising contacting a cancer cell with a combination comprising a 1,2-diphenylpyrrole derivative and an EGFR inhibitor whereby the combination inhibits proliferation of cancer cells. In one embodiment, the invention provides a method of inhibiting proliferation of cancer cells, the method comprising contacting a cancer cell with a combination comprising a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib.

In another embodiment the invention provides a method for reducing proliferation of cancer cells, the method comprising delivering to the cells a combination comprising a 1,2-diphenylpyrrole derivative and an EGFR inhibitor, whereby the reduction of cell proliferation is greater than a reduction caused by either a 1,2-diphenylpyrrole derivative alone or an EGFR inhibitor alone. In one embodiment, the invention provides a method for reducing proliferation of cancer cells, the method comprising delivering to the cells a combination comprising a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib.

In one embodiment the invention provides a method of modulating autophosphorylation with a molecule of ATP, the method comprising delivering to a cancer cell an effective amount of a combination comprising a 1,2-diphenylpyrrole derivative and an EGFR inhibitor wherein the combination inhibits autophosphorylation with a molecule of ATP. In one embodiment, the invention provides a method of modulating autophosphorylation with a molecule of ATP, the method comprising delivering to a cancer cell an effective amount of a combination comprising a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib.

In a further embodiment the invention provides a method of inhibiting metastases of tumor cells, the method comprising administering an effective amount of a combination comprising a 1,2-diphenylpyrrole derivative and an EGFR inhibitor such that the combination inhibits metastatic activity of tumor cells. In one embodiment, the invention provides a method of inhibiting metastases of tumor cells, the method comprising administering an effective amount of a combination comprising a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib.

In one embodiment the invention provides a method for inducing apoptosis in cancer cells, the method comprising contacting the cancer cells with a combination comprising a 1,2-diphenylpyrrole derivative and an EGFR inhibitor sufficient to induce apoptosis. In one embodiment, the invention provides a method for inducing apoptosis in cancer cells, the method comprising contacting the cancer cells with a combination comprising a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib.

In another embodiment the invention provides a method for sensitizing EGFR inhibitor resistant cancer cells to an EGFR inhibitor, the method comprising administering a combination comprising a 1,2-diphenylpyrrole derivative and an EGFR inhibitor wherein the combination sensitizes the cancer cells to the EGFR inhibitor. In one embodiment, the invention provides a method for sensitizing EGFR inhibitor resistant cancer cells to an EGFR inhibitor, the method comprising administering a combination comprising a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib.

In a further embodiment the invention provides a method of modulating prostaglandin synthesis in a cancer cell, the method comprising contacting the cell with a combination comprising a 1,2-diphenylpyrrole derivative and an EGFR inhibitor wherein the combination inhibits prostaglandin synthesis in a cancer cell. In one embodiment, the invention provides a method of modulating prostaglandin synthesis in a cancer cell, the method comprising contacting the cell with a combination comprising a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib.

In one embodiment the invention provides a method of modulating cyclooxygenase expression in a cancer cell, the method comprising delivering to the cell a combination comprising a 1,2-diphenylpyrrole derivative and an EGFR inhibitor wherein the combination inhibits cyclooxygenase expression in a cancer cell. In one embodiment, the invention provides a method of modulating cyclooxygenase expression in a cancer cell, the method comprising delivering to the cell a combination comprising a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib.

In one embodiment the invention provides a method of modulating angiogenesis in a cancer cell, the method comprising contacting the cell with a combination comprising a 1,2-diphenylpyrrole derivative and an EGFR inhibitor wherein the combination inhibits angiogenesis in a cancer cell. In one embodiment the invention provides a method of modulating angiogenesis in a cancer cell, the method comprising contacting the cell with a combination comprising a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib. In another embodiment the invention provides a method of reducing the dosage in conventional treatment for neoplasia and/or neoplasia related disorders in a subject, the method comprising administering to a subject a combination of a 1,2-diphenylpyrrole derivative and an EGFR inhibitor wherein the combination reduces the dosage in conventional treatment for neoplasia and/or neoplasia-related disorders. In one embodiment, the invention provides a method of reducing the dosage in conventional treatment for neoplasia and/or neoplasia related disorders in a subject, the method comprising administering to a subject a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib.

In one embodiment the invention provides a method of treating neoplasia and/or neoplasia related disorders, the method comprising administering a combination of a 1,2-diphenylpyrrole derivative and an EGFR inhibitor. In one embodiment, embodiment the invention provides a method of treating neoplasia and/or neoplasia related disorders, the method comprising administering a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib.

The combinations presently described herein may also be useful in the treatment of additional disorders in which aberrant expression ligand/receptor interactions or activation or signalling events related to various protein tyrosine kinases are involved. Such disorders may include those of neuronal, glial, astrocytal, hypothalamic, glandular, macrophagal, epithelial, stromal, or blastocoelic nature in which aberrant function, expression, activation or signalling of the erbB tyrosine kinases are involved. In addition, the combinations presented herein may have therapeutic utility in inflammatory, angiogenic and immunologic disorders involving both identified and as yet unidentified tyrosine kinases that are inhibited by the combinations presented herein.

### Combination Therapy

In some embodiments, the composition comprising a combination of a 1,2-diphenylpyrrole derivative and an EGFR inhibitor described herein, has an effect that is additive of the effects of the 1,2-diphenylpyrrole derivative alone and the effects of the EGFR inhibitor alone. In another embodiment, the invention provides a composition comprising, a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib, wherein the combination has an effect that is additive of the effects of the 2-(4-ethoxyphenyl)-4-methyl-1-(4-sulfamoylphenyl)-pyrrole alone and the effects of erlotinib alone.

In some other embodiments, the composition comprising a combination of a 1,2-diphenylpyrrole derivative and an EGFR inhibitor described herein, has an effect that is greater than the additive effects of the 1,2-diphenylpyrrole derivative alone and the effects of the EGFR inhibitor alone. In another embodiment, the invention provides a composition comprising, a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib, wherein the combination has an effect that is greater than the additive effects of the 2-(4-ethoxyphenyl)-4-methyl-1-(4-sulfamoylphenyl)-pyrrole alone and the effects of erlotinib alone.

In some embodiments, the composition comprising a combination of a 1,2-diphenylpyrrole derivative and an EGFR inhibitor described herein, has an effect that is greater than the effects of the 1,2-diphenylpyrrole derivative alone (e.g., cyclooxygenase-2 inhibition alone). In another embodiment, the invention provides a composition comprising, a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib, wherein the combination has an effect that is greater than the effects of the 2-(4-ethoxyphenyl)-4-methyl-1-(4-sulfamoylphenyl)-pyrrole alone.

In other embodiments, the composition comprising a combination of a 1,2-diphenylpyrrole derivative and an EGFR inhibitor described herein, has an effect that is greater than the effects of the EGFR inhibitor alone (epidermal growth factor receptor kinase inhibition alone). In another embodiment, the invention provides a composition comprising, a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib, wherein the combination has an effect that is greater than the effects of erlotinib alone.

In other embodiments, the invention provides a method for treating cancer, tumors, and tumor-related disorders comprising administering a composition comprising a combination of a 1,2-diphenylpyrrole derivative and an EGFR inhibitor described herein, wherein the combination has an effect that is additive of the effects of the 1,2-diphenylpyrrole derivative alone and the effects of the EGFR inhibitor alone. In further embodiments, the invention provides a method for treating cancer, tumors, and tumor-related disorders comprising administering a composition comprising a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib, wherein the combination has an effect that is additive of the effects of 2-(4-ethoxyphenyl)-4-methyl-1-(4-sulfamoylphenyl)-pyrrole alone and the effects of erlotinib alone.

In some other embodiments, the invention provides a method for treating cancer, tumors, and tumor-related disorders, comprising administering a composition comprising a combination of a 1,2-diphenylpyrrole derivative and an EGFR inhibitor described herein, wherein the combination has an effect that is greater than the additive effects of the effects of the 1,2-diphenylpyrrole derivative alone and the effects of the EGFR inhibitor alone. In other embodiments, the invention provides method for treating cancer, tumors, and tumor-related disorders, comprising administering a composition comprising a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib, wherein the combination has an effect that is greater than the additive effects of 2-(4-ethoxyphenyl)-4-methyl-1-(4-sulfamoylphenyl)-pyrrole alone and the effects of erlotinib alone.

In some embodiments, the invention provides a method for treating cancer, tumors, and tumor-related disorders comprising administering a composition comprising a combination of a 1,2-diphenylpyrrole derivative and an EGFR inhibitor described herein, wherein the combination has an effect that is greater than the effects of the 1,2-diphenylpyrrole derivative alone (e.g., cyclooxygenase-2 inhibition alone). In other embodiments, the invention provides a method for treating cancer, tumors, and tumor-related disorders comprising administering a composition comprising a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib, wherein the combination has an effect that is greater than the effects of is 2-(4-ethoxyphenyl)-4-methyl-1-(4-sulfamoylphenyl)-pyrrole alone.

In further embodiments, the invention provides a method for treating cancer, tumors, and tumor-related disorders comprising administering a composition comprising a combination of a 1,2-diphenylpyrrole derivative and an EGFR inhibitor described herein, wherein the combination has an effect that is greater than the effects of the EGFR inhibitor alone (epidermal growth factor receptor kinase inhibition alone). In other embodiments, the invention provides a method for treating cancer, tumors, and tumor-related disorders comprising administering a composition comprising a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib, wherein the combination has an effect that is greater than the effects of erlotinib alone.

Synergism of the composition comprising a combination of a 1,2-diphenylpyrrole derivative and an EGFR inhibitor, may be used to obtain the desired effect at doses to which side effects are minimal. For example, a patient may be treated for a disease, disorder, or condition which benefits from EGFR inhibition, such as tumors, tumor-related diseases, cancer, neoplasia, while concomitantly being treated for a side effect of the EGFR inhibition, such as inflammation, through the benefit of the 1,2-diphenylpyrrole derivative inhibitor. In one embodiment, the invention provides a combination of 2-(4-ethoxyphenyl)-4-methyl-1-(4-sulfamoylphenyl)-pyrrole and erlotinib which may be used to obtain the desired effect at doses to which side effects are minimal.

The composition comprising a combination of a 1,2-diphenylpyrrole derivative and an EGFR inhibitor, may be applied as a sole therapy or may involve one or more other materials and treatment agents such as but not limited to a combination of inhibitors of MMP (matrix-metallo-proteinase), other tyrosine kinases including VEGFR (vascular endothelial growth factor receptor), farnesyl transferase, CTLA₄ (cytotoxic T-lymphocyte antigen 4) and erbB2, as well as MAb to VEGFr, and other cancer-related antibodies including rhuMAb-VEGF, the erbB2 MAb, or avb3.

Thus, the composition comprising a combination of a 1,2-diphenylpyrrole derivative and an EGFR inhibitor, may be applied with one or more other anti-tumor substances, for example, those selected from, mitotic inhibitors, for example vinblastine; alkylating agents, for example, cis-platin, carboplatin, and cyclophosphamide; anti-metabolites, for example 5-fluorouracil, cytosine arabinoside and hydroxyurea, or, for example, anti-metabolites such as N-(5-[N-(3,4-dihydro-2-methyl-4-oxoquinazolin-6-ylmethyl)-N-methylamino]-2-thenyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex® (tamoxifen) or, for example anti-androgens such as Casodex® (4′-cyano-3-(4-fluorophenyl sulphonyl)-2-hydroxy-2-methyl-3′-(trifluoromethyl)propionanilide). In one embodiment, the invention provides a combination a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib, may be applied with one or more other anti-tumor substances, for example, those selected from, mitotic inhibitors, for example vinblastine; alkylating agents, for example, cis-platin, carboplatin, and cyclophosphamide; anti-metabolites, for example 5-fluorouracil, cytosine arabinoside and hydroxyurea, or, for example, anti-metabolites such as N-(5-[N-(3,4-dihydro-2-methyl-4-oxoquinazolin-6-ylmethyl)-N-methylamino]-2-thenyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex® (tamoxifen) or, for example anti-androgens such as Casodex® (4′-cyano-3-(4-fluorophenyl sulphonyl)-2-hydroxy-2-methyl-3′-(trifluoromethyl)propionanilide).

For the combination therapies including combination therapies having pharmaceutical compositions described herein, the effective amounts of the compound presently described herein useful for inhibiting abnormal cell growth (e.g., other antiproliferative agent, anti-angiogenic, signal transduction inhibitor or immune-system enhancer) can be determined by those of ordinary skill in the art, based on the effective amounts for the compound described herein and those known or described for the chemotherapeutic or other agent. The formulations and routes of administration for such therapies and compositions can be based on the information described herein for compositions and therapies comprising the combinations presented herein as the sole active agent and on information provided for the chemotherapeutic or other agent in combination therewith.

In one embodiment, the invention provides a method for inhibiting abnormal cell growth in a subject comprising administering to the subject an effective amount of a composition comprising a combination of a 1,2-diphenylpyrrole derivative and an EGFR inhibitor, or their pharmaceutically acceptable salt, solvate or prodrug thereof, in combination with radiation therapy effective in inhibiting abnormal cell growth in the subject. Techniques for administering radiation therapy are known to a person of skill in the art and these techniques can be used in the combination therapy described herein.

### EGFR Resistance

Studies have shown that after a period of about 8-12 months of EGFR-directed treatments, the cancer cells become resistant to the treatment, most commonly by 1) recruiting a mutated IGF-1 receptor to act as one of the EGFR partners in the homodimer, thus, forming a heterodimer (this allows the signal to be transmitted even in the presence of an EGFR inhibitor); 2) the presence of redundant tyrosine kinase receptors; 3) increased angiogenesis; 4) the constitutive activation of downstream mediators; and 5) the existence of specific EGFR mutations. Understanding the molecular mechanisms of resistance and sensitivity may lead to improvements in therapies that target EGFR.

IGF-1R activates many of the same downstream pathways as EGFR and can lead to tumorigenesis, increased proliferation, angiogenesis, and metastasis. PI-3K/Akt signaling is a critical component of the downstream mediation of EGFR and also plays a functional role in IGF-1R signaling. Without wishing to be bound by any particular theory, this redundancy may explain how the receptors can mimic the function of one another. Chakravarti et al., identified two glioblastoma cell lines that each overexpressed EGFR but exhibited very different responses to EGFR inhibitors. The resistant cell line significantly overexpressed IGF-1R and showed further increases in IGF-1R expression in response to EGFR inhibition by AG1478, an EGFR TKI. PI-3K/Akt signaling persisted in these resistant cell lines in response to AG1478 treatment, and these cells also maintained their invasive and antiapoptotic characteristics. These studies support the concept of redundant signaling through IGF-IR that maintains activation of critical pathways for survival in the presence of EGFR inhibition. Inhibiting both IGF-1R and EGFR may significantly reduce the growth and invasiveness of cells that are resistant to EGFR inhibitors alone. Further evidence that IGF-1R activation may bypass inhibition of other tyrosine kinase receptors comes from a study by Lu et al., who showed that the degree of overexpression of IGF-1R was inversely related to the response of breast cancer cells to trastuzumab, an antibody directed against ErbB2. SKBR3 human breast cancer cells, which normally overexpress HER2/neu and minimally express IGF-1R, showed a 42% decrease in proliferation in response to trastuzumab. Unlike the parental cell line, SKBR3 cells that were engineered to overexpress IGF-1R showed no response to trastuzumab. When the IGF-1R was inhibited by IGF-binding protein-3 in the engineered cell lines, the response to trastuzumab returned to normal. These studies clearly indicate that activation of alternative tyrosine kinase receptors in tumor cells may override the effect of EGFR family inhibitors. These examples suggest that a combination may be able to overcome resistance to a single receptor inhibitor and thus more effectively inhibit pathways leading to cancer growth and survival. Recently, an enhanced response was shown in breast cancer cell lines treated with either recombinant bispecific antibodies to both EGFR and IGF-1R or a combination of single receptor antibodies compared with either antibody alone. Signaling pathways downstream of the receptors were also more effectively inhibited by the combination therapy. By combining therapies that attack multiple cell surface and intracellular signaling pathways, redundant receptor signaling might be blocked and greater clinical benefit achieved. Thus, identifying key downstream signaling molecules in which growth factor receptor signals converge may be important in the development of therapeutic agents that block signals from multiple activated growth factor receptors. Similarly, Jung et al. discloses the use of a combination of mAbs to EGFR and mAbs to VEGFR-2 to treat gastric cancer grown in nude mice. Both mAbs were modestly effective at inhibiting tumor growth, but the combination achieved significantly greater tumor growth inhibition that was also associated with decreased tumor vascularity and increased tumor cell apoptosis.

In one embodiment the invention provides a method for treating a subject having an EGFR inhibitor resistant cancer cell comprising administering to the subject a therapeutically effective amount of a composition comprising a 1,2-diphenylpyrrole derivative in combination with an EGFR inhibitor. In one embodiment, the 1,2-diphenylpyrrole derivative is 2-(4-ethoxyphenyl)-4-methyl-1-(4-sulfamoylphenyl)-pyrrole. In another embodiment the 1,2-diphenylpyrrole derivative is 2-(4-ethoxyphenyl)-4-methyl-1-(4-sulfamoylphenyl)-pyrrole and the EGFR inhibitor is erlotinib.

In one embodiment the invention provides a method for sensitizing EGFR inhibitor resistant cancer cells to an EGFR inhibitor, the method comprising administering a combination comprising a 1,2-diphenylpyrrole derivative and an EGFR inhibitor wherein the combination sensitizes the cancer cells to the EGFR inhibitor. In one embodiment, the invention provides a method for sensitizing EGFR inhibitor resistant cancer cells to erlotinib, the method comprising administering a combination comprising a 2-(4-ethoxyphenyl)-4-methyl-1-(4-sulfamoylphenyl)-pyrrole and erlotinib wherein the combination sensitizes the cancer cells erlotinib.

### Lung Cancer

In many countries including Japan, Europe and America, the number of patients with lung cancer is fairly large and continues to increase year after year and is the most frequent cause of cancer death in both men and women. Although there are many potential causes for lung cancer, tobacco use, and particularly cigarette smoking, is the most important. Additionally, etiologic factors such as exposure to asbestos, especially in smokers, or radon are contributory factors. Also occupational hazards such as exposure to uranium have been identified as an important factor. Finally, genetic factors have also been identified as another factor that increases the risk of cancer.

Lung cancers can be histologically classified into non-small cell lung cancers (e.g. squamous cell carcinoma (epidermoid), adenocarcinoma, large cell carcinoma (large cell anaplastic), etc.) and small cell lung cancer (oat cell). Non-small cell lung cancer (NSCLC) has different biological properties and responses to chemotherapeutics from those of small cell lung cancer (SCLC). Thus, chemotherapeutic formulas and radiation therapy are different between these two types of lung cancer.

### Non-Small Cell Lung Cancer

Where the location of the non-small cell lung cancer tumor can be easily excised (stage I and II disease) surgery is the first line of therapy and offers a relatively good chance for a cure. In more advanced disease (stage IIIa and greater), however, where the tumor has extended to tissue beyond the bronchopulmonary lymph nodes, surgery may not lead to complete excision of the tumor. In such cases, the patient's chance for a cure by surgery alone is greatly diminished. Where surgery will not provide complete removal of the NSCLC tumor, other types of therapies must be utilized. Today, chemoradiation therapy, is the standard treatment to control unresectable or inoperable NSCLC. Improved results have been seen when radiation therapy has been combined with chemotherapy, but gains have been modest and the search continues for improved methods of combining modalities.

Radiation therapy is based on the principle that high-dose radiation delivered to a target area will result in the death of reproductive cells in both tumor and normal tissues. The radiation dosage regimen is generally defined in terms of radiation absorbed dose (rad), time and fractionation, and must be carefully defined by the oncologist. The amount of radiation a patient receives will depend on various consideration but the two most important considerations are the location of the tumor in relation to other critical structures or organs of the body, and the extent to which the tumor has spread. One course of treatment for a patient undergoing radiation therapy for NSCLC will be a treatment schedule over a 5 to 6 week period, with a total dose of 50 to 60 Gy administered to the patient in a single daily fraction of L8 to 2.0 Gy, 5 days a week. A Gy is an abbreviation for Gray and refers to 100 rad of dose.

As NSCLC is a systemic disease, however, and radiation therapy is a local modality, radiation therapy as a single line of therapy is unlikely to provide a cure for NSCLC, at least for those tumors that have metastasized distantly outside the zone of treatment. Thus, the use of radiation therapy with other modality regimens has important beneficial effects for the treatment of NSCLC.

Generally, radiation therapy has been combined temporally with chemotherapy to improve the outcome of treatment. There are various terms to describe the temporal relationship of administering radiation therapy in combination with COX-2 inhibitors and chemotherapy, and the following examples are the treatment regimens and are provided for illustration only and are not intended to limit the use of other combinations. “Sequential” therapy refers to the administration of a composition comprising a combination of 2-(4-ethoxyphenyl)-4-methyl-1-(4-sulfamoylphenyl)-pyrrole and erlotinib and/or radiation therapy separately in time in order to allow the separate administration of the composition, and/or radiation therapy. “Concomitant” therapy refers to the administration of the composition, and/or radiation therapy on the same day.

Finally, “alternating” therapy refers to the administration of radiation therapy on the days in which the composition would not have been administered if it was given alone. It is reported that advanced non-small cell lung cancers do not respond favorably to single-agent chemotherapy and useful therapies for advanced inoperable cancers have been limited. (Journal of Clinical Oncology, vol. 10, pp. 829-838 (1992)).

Japanese Patent Kokai 5-163293 refers to some specified antibiotics of 16-membered-ring macrolides as a drug delivery carrier capable of transporting anthracycline-type anticancer drugs into the lungs for the treatment of lung cancers. Thus, the use of a composition comprising a combination of 2-(4-ethoxyphenyl)-4-methyl-1-(4-sulfamoylphenyl)-pyrrole and erlotinib and a macrolide as a drug delivery carrier is contemplated herein for the treatment of non-small cell lung cancer.

Several chemotherapeutic agents have been shown to be efficacious against NSCLC. Chemotherapeutic agents that can be used in the present disclosure against NSCLC include cisplatin, carboplatin, paclitaxel, docetaxel, taxane formulations such as by way of example only, Abraxane® (ABI-007), Paclitaxel-Cremophor EL, Paclitaxel poliglumex, and Paclitaxel injectable emulsion (PIE), gemcitabine, navelbine, pemetrexate, etoposide, methotrexate, 5-Fluorouracil, epirubicin, doxorubicin, vinblastine, cyclophosphamide, ifosfamide, mitomycin C, epirubicin, vindesine, camptothecins, fotemustine, and edatrexate.

In one embodiment, the invention provides a method of the treatment of NSCLC which utilizes a therapeutically effective amounts of a composition comprising a combination of 2-(4-ethoxyphenyl)-4-methyl-1-(4-sulfamoylphenyl)-pyrrole and erlotinib. Further, therapy for the treatment of NSCLC utilizes a therapeutically effective amounts of a composition comprising a combination of 2-(4-ethoxyphenyl)-4-methyl-1-(4-sulfamoylphenyl)-pyrrole with erlotinib, and one of the following antineoplastic agents: bevacizumab, docetaxel, gefitinib, gemcitabine, cisplatin, carboplatin, etoposide, paclitaxel, pemetrexate, vinorelbine, or radiation therapy.

### Small Cell Lung Cancer

Approximately 15 to 20 percent of all cases of lung cancer reported worldwide are small cell lung cancer (SCLC) (Ride DC: Cancer 54:2722, 1984). Currently, treatment of SCLC incorporates multi-modal therapy, including chemotherapy and radiation therapy. Response rates of localized or disseminated SCLC remain high to systemic chemotherapy, however, persistence of the primary tumor and persistence of the tumor in the associated lymph nodes has led to the integration of several therapeutic modalities in the treatment of SCLC.

Therapy for the treatment of lung cancer according to one embodiment of the invention utilizes a combination of therapeutically effective amount of a composition comprising a combination of 2-(4-ethoxyphenyl)-4-methyl-1-(4-sulfamoylphenyl)-pyrrole and erlotinib. In another embodiment, therapy for the treatment of lung cancer according to the invention utilizes a combination of therapeutically effective amount of a composition comprising a combination of 2-(4-ethoxyphenyl)-4-methyl-1-(4-sulfamoylphenyl)-pyrrole and erlotinib and one of the following antineoplastic agents: vincristine, docetaxel, camptothecin, topotecan cisplatin, carboplatin, cyclophosphamide, epirubicin (high dose), etoposide (VP-16) I.V., etoposide (VP-16) oral, isofamide, teniposide (VM-26), doxorubicin, and amrubicin. Other preferred single-agents chemotherapeutic agents that may be used in the present disclosure include BCNU (carmustine), vindesine, hexamethylmelamine (altretamine), methotrexate, nitrogen mustard, and CCNU (lomustine). Other chemotherapeutic agents under investigation that have shown activity against SCLC include iroplatin, gemcitabine, Ionidamine, and taxol.

### Glioma

A glioma is a type of primary central nervous system (CNS) tumor that arises from glial cells. The most common site of involvement of gliomas is the brain, but they can also affect the spinal cord or any other part of the CNS, such as the optic nerves. Treatment for brain gliomas depends on the location and the grade. Often, treatment is a combined approach, using surgery, radiation therapy, and chemotherapy. The radiation therapy is in the form of external beam radiation or the stereotactic approach using radiosurgery. Spinal cord tumors can be treated by surgery and radiation. Temozolomide is a chemotherapeutic drug that is able to cross the blood-brain barrier effectively and is being used in therapy.

EGFR is frequently amplified, overexpressed, or mutated in glioblastomas, but generally only 10 to 20% of patients have a response to EGFR inhibitors. It has been shown that glioblastoma that overexpresses the EGFRvIII oncogene and are PTEN tumor suppressor wildtype are sensitive to erlotinib treatment.

Therapy for the treatment of glioma according to one embodiment of the invention utilizes a combination of therapeutically effective amount of a composition comprising a combination of 2-(4-ethoxyphenyl)-4-methyl-1-(4-sulfamoylphenyl)-pyrrole and erlotinib. In another embodiment, therapy for the treatment of glioma according to the invention utilizes a combination of therapeutically effective amount of a composition comprising a combination of 2-(4-ethoxyphenyl)-4-methyl-1-(4-sulfamoylphenyl)-pyrrole and erlotinib and temozolomide.

### Head and Neck Cancer

Head and neck cancer includes cancers of the mouth, nose, sinuses, salivary glands, throat and lymph nodes in the neck. Most begin in the moist tissues that line the mouth, nose and throat. Symptoms include a lump or sore that does not heal, a sore throat that does not go away, trouble swallowing and a change or hoarseness in the voice. Using tobacco or alcohol increases cancer risk. Treatments of head and neck cancer may include surgery, radiation therapy, chemotherapy or a combination. Treatments can affect eating, speaking or even breathing, so patients may need rehabilitation.

Head and neck cancer is often complex, with many different sites and staging systems. However, current therapy offers several alternatives, including surgery, radiation, and chemotherapy, either alone or in combination.

Combined modality therapy is becoming the principal method of treating patients with locally advanced head and neck cancers. Meanwhile, researchers are actively investigating new treatments such as gene therapy. Newer chemotherapy agents (e.g., paclitaxel [Taxol®], docetaxel [Taxotere®], gemcitabine [Gemzar®], doxorubicin [Doxil®]) may be combined with established chemotherapeutic agents (e.g., methotrexate [Trexall®, Methotrex®]) to improve results.

In March 2006, the Food and Drug Administration (FDA) approved cetuximab (Erbitux®), in combination with radiation, for patients with squamous cell carcinoma of the head and neck that cannot be treated surgically. Cetuximab also may be used alone (called monotherapy) in patients with head and neck cancer that has spread (metastasized) following standard chemotherapy.

Therapy for the treatment of head and neck cancer according to one embodiment of the invention utilizes a combination of therapeutically effective amount of a composition comprising a combination of 2-(4-ethoxyphenyl)-4-methyl-1-(4-sulfamoylphenyl)-pyrrole and erlotinib. In another embodiment, therapy for the treatment of head and neck cancer according to the invention utilizes a combination of therapeutically effective amount of a composition comprising a combination of 2-(4-ethoxyphenyl)-4-methyl-1-(4-sulfamoylphenyl)-pyrrole and erlotinib and one or more chemotherapy agents (e.g., paclitaxel [Taxol®], docetaxel [Taxotere®], gemcitabine [Gemzar®], doxorubicin [Doxil®]) which may be further combined with established chemotherapeutic agents (e.g., methotrexate [Trexall®, Methotrex®]). In another embodiment, therapy for the treatment of head and neck cancer according to the invention utilizes a combination of therapeutically effective amount of a composition comprising a combination of 2-(4-ethoxyphenyl)-4-methyl-1-(4-sulfamoylphenyl)-pyrrole and erlotinib and Erbitux®.

### Colorectal Cancer

COX-2 expression levels correlate with survival rates in colorectal cancer. Survival from colorectal cancer depends on the stage and grade of the tumor, for example precursor adenomas to metastatic adenocarcinoma. Generally, colorectal cancer can be treated by surgically removing the tumor, but overall survival rates remain between 45 and 60 percent. Colonic excision morbidity rates are fairly low and are generally associated with the anastomosis and not the extent of the removal of the tumor and local tissue. In patients with a high risk of reoccurrence, however, chemotherapy has been incorporated into the treatment regimen in order to improve survival rates.

Tumor metastasis prior to surgery is generally believed to be the cause of surgical intervention failure and up to one year of chemotherapy is required to kill the non-excised tumor cells. As severe toxicity is associated with the chemotherapeutic agents, only patients at high risk of recurrence are placed on chemotherapy following surgery. Thus, the incorporation of an antiangiogenesis inhibitor into the management of colorectal cancer plays an important role in the treatment of colorectal cancer and lead to overall improved survival rates for patients diagnosed with colorectal cancer.

One embodiment of the invention provides a combination therapy for the treatment of colorectal cancer including surgery, followed by a regimen of a composition comprising a combination of 2-(4-ethoxyphenyl)-4-methyl-1-(4-sulfamoylphenyl)-pyrrole and erlotinib. Further, another embodiment of the invention provides a combination therapy for the treatment of colorectal cancer including surgery, followed by a regimen of a composition comprising a combination of 2-(4-ethoxyphenyl)-4-methyl-1-(4-sulfamoylphenyl)-pyrrole and erlotinib, and one or more antiangiogenic agents including an MMP inhibitor, or an integrin antagonist, cycled over a one year time period. A further embodiment of the invention provides a combination therapy for the treatment of colorectal cancer including a regimen of a composition comprising a combination of 2-(4-ethoxyphenyl)-4-methyl-1-(4-sulfamoylphenyl)-pyrrole and erlotinib, followed by surgical removal of the tumor from the colon or rectum and then followed be a regimen of a composition comprising a combination of 2-(4-ethoxyphenyl)-4-methyl-1-(4-sulfamoylphenyl)-pyrrole and erlotinib cycled over a one year time period. Another therapy for the treatment of colon cancer comprises administering a combination of therapeutically effective amounts of a composition comprising a combination of 2-(4-ethoxyphenyl)-4-methyl-1-(4-sulfamoylphenyl)-pyrrole and erlotinib.

A further therapy for the treatment of colon cancer is a combination of a treatment with a composition comprising a combination of 2-(4-ethoxyphenyl)-4-methyl-1-(4-sulfamoylphenyl)-pyrrole and erlotinib in combination with the following antineoplastic agents: fluorouracil, Levamisole, camptothecin, oxaliplatin, bevacizumab, cetuximab, panitumumab, irinotecan, leucovorin, and capecitabine.

### Breast Cancer

Today, among women in the United States, breast cancer remains the most frequent diagnosed cancer. One in 8 women in the United States is at risk of developing breast cancer in their lifetime. Age, family history, diet, and genetic factors have been identified as risk factors for breast cancer. Breast cancer is the second leading cause of death among women.

Different chemotherapeutic agents are known in art for treating breast cancer. Cytoxic agents used for treating breast cancer include doxorubicin, cyclophosphamide, methotrexate, 5-fluorouracil, mitomycin C, mitoxantrone, paclitaxel, taxane formulations such as by way of example only, Abraxane® (ABI-007), Paclitaxel-Cremophor EL, Paclitaxel poliglumex, and Paclitaxel injectable emulsion (PIE), gemcitabine, docetaxel, capecitabine, lapatanib, trastuzumab, anastrozole, letrozole, exemestane, and epirubicin. In the treatment of locally advanced noninflammatory breast cancer, a composition comprising a combination of 2-(4-ethoxyphenyl)-4-methyl-1-(4-sulfamoylphenyl)-pyrrole and erlotinib can be used to treat the disease. Additionally, in combination with surgery, radiation therapy or with chemotherapeutic or other antiangiogenic agents. Other combinations of chemotherapeutic agents, that can be used in combination with the present disclosure include, but are not limited to anastrozole, capecitabine, docetaxel, epirubicin, exemestane, fulvestrant, epothilone A, B or D, goserelin acetate, letrozole, bevacizumab, paclitaxel, pamidronate, tamoxifen, toremifene, and trastuzumab.

Hormone Positive

Many breast cancers require the hormone oestrogen to grow. In women who have had their menopause, the main source of oestrogen is through the conversion of androgens into oestrogens. This process is carried out by the aromatase enzyme. In the treatment of hormone positive breast cancer a composition comprising a combination of 2-(4-ethoxyphenyl)-4-methyl-1-(4-sulfamoylphenyl)-pyrrole and erlotinib can be used to treat the disease in combination with other agents, such as, aromatase inhibitors, for e.g., exemestane, letrozole, and anastrozole.

HER2/neu Positive Breast Cancer

In the treatment of HER2/neu positive breast cancer, compositions comprising a combination of 2-(4-ethoxyphenyl)-4-methyl-1-(4-sulfamoylphenyl)-pyrrole and erlotinib can be used to treat the disease in combination with other antiangiogenic agents, or in combination with surgery, radiation therapy or with chemotherapeutic agents. Other chemotherapeutic agents include, for example, trastuzumab, lapatinib, and CL-387785.

Triple Negative Breast Cancer

In the treatment of triple negative breast cancer wherein the cancer is estrogen receptor-negative, progesterone receptor-negative and HER2-negative, compositions comprising a combination of 244-ethoxyphenyl)-4-methyl-1-(4-sulfamoylphenyl)-pyrrole and erlotinib can be used to treat the disease in combination with other therapeutic agents. Such agents include, by way of example only, cetuximab, paclitaxel, docetaxel, taxane formulations, for example, Abraxane® (ABI-007), Paclitaxel-Cremophor EL, Paclitaxel poliglumex, and Paclitaxel injectable emulsion (PIE).

### Ovarian Cancer

Celomic epithelial carcinoma accounts for approximately 90% of ovarian cancer cases. One therapy for the treatment of ovary cancer is a combination of therapeutically effective amounts of a composition comprising a combination of 2-(4-ethoxyphenyl)-4-methyl-1-(4-sulfamoylphenyl)-pyrrole and erlotinib.

Other agents that can be used in combination with a composition comprising a combination 2-(4-ethoxyphenyl)-4-methyl-1-(4-sulfamoylphenyl)-pyrrole and erlotinib include, but are not limited to: alkylating agents, ifosfamide, cisplatin, carboplatin, paclitaxel, docetaxel, PEGylated liposomal doxorubicin, gemcitabine, doxorubicin, epothilone A, B, or D, topotecan, liposomal doxorubicin 5-fluorouracil, methotrexate, mitomycin, hexamethylmelamine, progestins, antiestrogens, prednimustine, dihydroxybusulfan, galactitol, interferon alpha, and interferon gam.

Other combinations for the treatment of celomic epithelial carcinoma is a combination of therapeutically effective amounts of a composition comprising a combination of 2-(4-ethoxyphenyl)-4-methyl-1-(4-sulfamoylphenyl)-pyrrole and erlotinib and one of the following combinations of antineoplastic agents: 1) cisplatin, doxorubicin, cyclophosphamide; 2) hexamthylmelamine, cyclosphamide, doxorubicin, cisplatin; 3) cyclophosphamide, hexamethylmelamine, 5-fluorouracil, cisplatin; 4) melphalan, hexamethylmelamine, cyclophosphamide; 5) melphalan, doxorubicin, cyclophosphamide; 6) cyclophosphamide, cisplatin, carboplatin; 7) cyclophosphamide, doxorubicin, hexamethylmelamine, cisplatin; 8) cyclophosphamide, doxorubicin, hexamethylmelamine, carboplatin; 9) cyclophosphamide, cisplatin; 10) hexamethylmelamine, doxorubicin, carboplatin; 11) cyclophosphamide, hexamethimelamine, doxorubicin, cisplatin; 12) carboplatin, cyclophosphamide; 13) cisplatin, cyclophosphamide.

Cancer of the fallopian tube accounts for approximately 400 new cancer cases per year in the United States. One therapy according to the invention is for the treatment of fallopian tube cancer which includes administering a combination of therapeutically effective amount of a composition comprising a combination of 2-(4-ethoxyphenyl)-4-methyl-1-(4-sulfamoylphenyl)-pyrrole and erlotinib. Other agents that can be used in combination with a composition comprising a combination 2-(4-ethoxyphenyl)-4-methyl-1-(4-sulfamoylphenyl)-pyrrole and erlotinib include, but are not limited to: alkylating agents, ifosfamide, cisplatin, carboplatin, paclitaxel, docetaxel, PEGylated liposomal doxorubicin, gemcitabine, doxorubicin, epothilone A, B, or D, topotecan, liposomal doxorubicin, 5-fluorouracil, methotrexate, mitomycin, hexamethylmelamine, progestins, antiestrogens, prednimustine, dihydroxybusulfan, galactitol, interferon α, and interferon γ.

Germ cell ovarian cancer accounts for approximately 5% of ovarian cancer cases. Germ cell ovarian carcinomas are classified into two main groups: dysgerminoma, and nondysgerminoma. Nondysgerminoma is further classified into teratoma, endodermal sinus tumor, embryonal carcinoma, chloricarcinoma, polyembryoma, and mixed cell tumors. One therapy for the treatment of germ cell carcinoma is a combination of therapeutically effective amount of a composition comprising a combination of 2-(4-ethoxyphenyl)-4-methyl-1-(4-sulfamoylphenyl)-pyrrole and erlotinib.

One therapy according to the invention is for the treatment of germ cell carcinoma which comprises administering a combination of a therapeutically effective amount of a composition comprising a combination of 2-(4-ethoxyphenyl)-4-methyl-1-(4-sulfamoylphenyl)-pyrrole and erlotinib and the following combinations of antineoplastic agents: 1) vincristine, actinomycin D, cyclophosphamide; 2) bleomycin, etoposide, cisplatin; 3) vinblastine, bleomycin, cisplatin.

### Pancreatic Cancer

Approximately 2% of new cancer cases diagnosed in the United States are pancreatic cancer. Pancreatic cancer is generally classified into two clinical types: 1) adenocarcinoma (metastatic and non-metastatic), and 2) cystic neoplasms (serous cystadenomas, mucinous cystic neoplasms, papillary cystic neoplasms, acinar cell systadenocarcinoma, cystic choriocarcinoma, cystic teratomas, angiomatous neoplasms).

Combinations of therapy for the treatment of non-metastatic adenocarcinoma that may be used in the present disclosure include the use of a composition comprising a combination of 2-(4-ethoxyphenyl)-4-methyl-1-(4-sulfamoylphenyl)-pyrrole and erlotinib along with preoperative bilary tract decompression (patients presenting with obstructive jaundice); surgical resection, including standard resection, extended or radial resection and distal pancreatectomy (tumors of body and tail); adjuvant radiation; antiangiogenic therapy; and chemotherapy.

For the treatment of metastatic adenocarcinoma, a combination therapy consists of a composition comprising a combination of 2-(4-ethoxyphenyl)-4-methyl-1-(4-sulfamoylphenyl)-pyrrole and erlotinib of the present disclosure in combination with continuous treatment of 5-fluorouracil, followed by weekly cisplatin therapy.

Another combination therapy for the treatment of cystic neoplasms is the use of a composition comprising a combination of 2-(4-ethoxyphenyl)-4-methyl-1-(4-sulfamoylphenyl)-pyrrole and erlotinib in combination with gemcitabine.

### Pharmaceutical Compositions

Provided herein is a pharmaceutical composition for treating cancer comprising a combination of a 1,2-diphenylpyrrole derivative and an EGFR inhibitor; and a pharmaceutically acceptable excipient or carrier.

In one embodiment, the invention provides a pharmaceutical composition comprising a combination of 2-(4-ethoxyphenyl)-4-methyl-1-(4-sulfamoylphenyl)-pyrrole and erlotinib as an active ingredient, or a pharmaceutically acceptable salt, solvate, or prodrug thereof, in a pharmaceutically acceptable vehicle, carrier, diluent, or excipient, or a mixture thereof; and one or more pharmaceutically acceptable excipients or carriers.

In another embodiment, the 1,2-diphenylpyrrole derivative is 2-(4-ethoxyphenyl)-4-methyl-1-(4-sulfamoylphenyl)-pyrrole and a pharmaceutically acceptable excipient or carrier. In another embodiment, the EGFR inhibitor is erlotinib and a pharmaceutically acceptable excipient or carrier. In a further embodiment the 1,2-diphenylpyrrole derivative is 2-(4-ethoxyphenyl)-4-methyl-1-(4-sulfamoylphenyl)-pyrrole and the EGFR inhibitor is erlotinib and a pharmaceutically acceptable excipient or carrier.

In one embodiment, the invention provides a pharmaceutical composition for treating cancer comprising a combination of a 1,2-diphenylpyrrole derivative selected from the group consisting of: 1,2-diphenylpyrrole derivative is selected from the group consisting of: 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole. In another embodiment, the invention provides a method wherein the 1,2-diphenylpyrrole derivative is 2-(4-ethoxyphenyl)-4-methyl-1-(4-sulfamoylphenyl)-pyrrole and an EGFR inhibitor, and a pharmaceutically acceptable excipient or carrier.

Provided herein are pharmaceutical compositions comprising a combination of a 1,2-diphenylpyrrole derivative and an EGFR inhibitor as an active ingredient, or a pharmaceutically acceptable salt, solvate, or prodrug thereof, in a pharmaceutically acceptable vehicle, carrier, diluent, or excipient, or a mixture thereof; and one or more pharmaceutically acceptable excipients or carriers. Also provided herein are pharmaceutical compositions comprising a combination of a 1,2-diphenylpyrrole derivative and an EGFR inhibitor as an active ingredient, or a pharmaceutically acceptable salt, solvate, or prodrug thereof, in a pharmaceutically acceptable vehicle, carrier, diluent, or excipient, or a mixture thereof; and one or more release controlling excipients as described herein. Provided herein are pharmaceutical compositions comprising a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib as an active ingredient, or a pharmaceutically acceptable salt, solvate, or prodrug thereof, in a pharmaceutically acceptable vehicle, carrier, diluent, or excipient, or a mixture thereof; and one or more release controlling excipients as described herein. Suitable modified release dosage vehicles include, but are not limited to, hydrophilic or hydrophobic matrix devices, water-soluble separating layer coatings, enteric coatings, osmotic devices, multi-particulate devices, and combinations thereof. The pharmaceutical compositions may also comprise non-release controlling excipients.

Provided herein are pharmaceutical compositions in film-coated dosage forms, which comprise a combination of a 1,2-diphenylpyrrole derivative and an EGFR inhibitor as an active ingredient, or a pharmaceutically acceptable salt, solvate, or prodrug thereof; and one or more tabletting excipients to form a tablet core using conventional tabletting processes and subsequently coating the core. The tablet cores can be produced using conventional granulation methods, for example wet or dry granulation, with optional comminution of the granules and with subsequent compression and coating. Granulation methods are described, for example, in Voigt, pages 156-69.

Suitable excipients for the production of granules are, for example pulverulent fillers optionally having flow-conditioning properties, for example talcum, silicon dioxide, for example synthetic amorphous anhydrous silicic acid of the Syloid® type (Grace), for example SYLOID 244 FP, microcrystalline cellulose, for example of the Avicel® type (FMC Corp.), for example of the types AVICEL PH101, 102, 105, RC581 or RC 591, Emcocel® type (Mendell Corp.) or Elcema® type (Degussa); carbohydrates, such as sugars, sugar alcohols, starches or starch derivatives, for example lactose, dextrose, saccharose, glucose, sorbitol, mannitol, xylitol, potato starch, maize starch, rice starch, wheat starch or amylopectin, tricalcium phosphate, calcium hydrogen phosphate or magnesium trisilicate; binders, such as gelatin, tragacanth, agar, alginic acid, cellulose ethers, for example methylcellulose, carboxymethylcellulose or hydroxypropylmethylcellulose, polyethylene glycols or ethylene oxide homopolymers, especially having a degree of polymerization of approximately from 2.0×10³ to 1.0×10³ and an approximate molecular weight of about from 1.0×10⁵ to 5.0×10⁶, for example excipients known by the name Polyox® (Union Carbide), polyvinylpyrrolidone or povidones, especially having a mean molecular weight of approximately 1000 and a degree of polymerization of approximately from about 500 to about 2500, and also agar or gelatin; surface-active substances, for example anionic surfactants of the alkyl sulfate type, for example sodium, potassium or magnesium n-dodecyl sulfate, n-tetradecyl sulfate, n-hexadecyl sulfate or n-octadecyl sulfate, of the alkyl ether sulfate type, for example sodium, potassium or magnesium n-dodecyloxyethyl sulfate, n-tetradecyloxyethyl sulfate, n-hexadecyloxyethyl sulfate or n-octadecyloxyethyl sulfate, or of the alkanesulfonate type, for example sodium, potassium or magnesium n-dodecanesulfonate, n-tetradecanesulfonate, n-hexadecanesulfonate or n-octadecanesulfonate, or non-ionic surfactants of the fatty acid polyhydroxy alcohol ester type, such as sorbitan monolaurate, monooleate, monostearate or monopalmitate, sorbitan tristearate or trioleate, polyoxyethylene adducts of fatty acid polyhydroxy alcohol esters, such as polyoxyethylene sorbitan monolaurate, monooleate, monostearate, monopalmitate, tristearate or trioleate, polyethylene glycol fatty acid esters, such as polyoxyethyl stearate, polyethylene glycol 400 stearate, polyethylene glycol 2000 stearate, especially ethylene oxide/propylene oxide block polymers of the Pluronics® (BWC) or Synperonic® (ICI) type

Further provided herein are pharmaceutical compositions in enteric coated dosage forms, which comprise a combination of a 1,2-diphenylpyrrole derivative and an EGFR inhibitor as an active ingredient, or a pharmaceutically acceptable salt, solvate, or prodrug thereof; and one or more release controlling excipients for use in an enteric coated dosage form. Provided herein are pharmaceutical compositions in enteric coated dosage forms comprising a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib as an active ingredient, or a pharmaceutically acceptable salt, solvate, or prodrug thereof, in a pharmaceutically acceptable vehicle, carrier, diluent, or excipient, or a mixture thereof; and one or more release controlling excipients for use in an enteric coated dosage form. The pharmaceutical compositions may also comprise non-release controlling excipients.

Further provided herein are pharmaceutical compositions in effervescent dosage forms, which comprise a combination of a 1,2-diphenylpyrrole derivative and an EGFR inhibitor as an active ingredient, or a pharmaceutically acceptable salt, solvate, or prodrug thereof; and one or more release controlling excipients for use in effervescent dosage forms. Also provided herein are pharmaceutical compositions in effervescent dosage forms comprising a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib as an active ingredient, or a pharmaceutically acceptable salt, solvate, or prodrug thereof, in a pharmaceutically acceptable vehicle, carrier, diluent, or excipient, or a mixture thereof; and one or more release controlling excipients for use in an effervescent dosage forms. The pharmaceutical compositions may also comprise non-release controlling excipients.

Additionally provided are pharmaceutical compositions in a dosage form that has an instant releasing component and at least one delayed releasing component, and is capable of giving a discontinuous release of the compound in the form of at least two consecutive pulses separated in time from 0.1 up to 24 hours. The pharmaceutical compositions comprise a combination of a 1,2-diphenylpyrrole derivative and an EGFR inhibitor as an active ingredient, or a pharmaceutically acceptable salt, solvate, or prodrug thereof; and one or more release controlling and non-release controlling excipients, such as those excipients suitable for a disruptable semi-permeable membrane and as swellable substances. Additionally, the invention provides pharmaceutical compositions comprising a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib as an active ingredient, or a pharmaceutically acceptable salt, solvate, or prodrug thereof; and one or more release controlling and non-release controlling excipients, such as those excipients suitable for a disruptable semi-permeable membrane and as swellable substances.

Provided herein also are pharmaceutical compositions in a dosage form for oral administration to a subject, which comprises a combination of a 1,2-diphenylpyrrole derivative and an EGFR inhibitor as an active ingredient, or a pharmaceutically acceptable salt, solvate, or prodrug thereof; and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer. Additionally, the invention provides pharmaceutical compositions in a dosage form for oral administration to a subject comprising a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib enclosed in an intermediate reactive layer comprising a gastric juice-resistant polymeric layered material partially neutralized with alkali and having cation exchange capacity and a gastric juice-resistant outer layer.

Provided herein are pharmaceutical compositions that comprise about 0.1 to about 1200 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 200 mg, about 300 mg, about 400 mg, about 500 mg, about 600 mg, about 800 mg, about 1200 mg of a combination of a 1,2-diphenylpyrrole derivative and an EGFR inhibitor as an active ingredient, or a pharmaceutically acceptable salt, solvate, or prodrug thereof, in the form of enteric-coated granules, as delayed-release capsules for oral administration. Also, the invention provides for pharmaceutical compositions that comprise about 0.1 to about 1000 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 500 mg of a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib as an active ingredient, or a pharmaceutically acceptable salt, solvate, or prodrug thereof, in the form of enteric-coated granules, as delayed-release capsules for oral administration.

Provided herein are pharmaceutical compositions that comprise about 0.1 to about 1200 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 500 mg, about 600 mg, about 800 mg, about 1200 mg of a combination of a 1,2-diphenylpyrrole derivative and an EGFR inhibitor as an active ingredient, or a pharmaceutically acceptable salt, solvate, or prodrug thereof in the form of enteric-coated pellets, as delayed-release capsules for oral administration. Also, the invention provides for pharmaceutical compositions that comprise about 0.1 to about 1200 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 500 mg, about 600 mg, about 800 mg, about 1200 mg of a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib as an active ingredient, or a pharmaceutically acceptable salt, solvate, or prodrug thereof in the form of enteric-coated pellets, as delayed-release capsules for oral administration. The pharmaceutical compositions further comprise glyceryl monostearate 40-50, hydroxypropyl cellulose, hypromellose, magnesium stearate, methacrylic acid copolymer type C, polysorbate 80, sugar spheres, talc, and triethyl citrate.

Provided herein are pharmaceutical compositions that comprise about 0.1 to about 1200 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 500 mg, about 600 mg, about 800 mg, about 1200 mg of a combination of a 1,2-diphenylpyrrole derivative and an EGFR inhibitor as an active ingredient, or a pharmaceutically acceptable salt, solvate, or prodrug thereof, as enteric-coated delayed-release tablets for oral administration. Also, the pharmaceutical compositions that comprise about 0.1 to about 1200 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 500 mg, about 600 mg, about 800 mg, about 1200 mg of a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib as an active ingredient, or a pharmaceutically acceptable salt, solvate, or prodrug thereof, as enteric-coated delayed-release tablets for oral administration. The pharmaceutical compositions further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

Provided herein are pharmaceutical compositions that comprise about 0.1 to about 1200 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 500 mg, about 600 mg, about 800 mg, about 1200 mg of a combination of a 1,2-diphenylpyrrole derivative and an EGFR inhibitor as an active ingredient, or a pharmaceutically acceptable salt, solvate, or prodrug thereof, as enteric-coated delayed-release tablets for oral administration. Also provided herein are pharmaceutical compositions that comprise about 0.1 to about 1200 mg, about 1 to about 500 mg, about 2 to about 100 mg, about 1 mg, about 2 mg, about 3 mg, about 5 mg, about 10 mg, about 20 mg, about 30 mg, about 40 mg, about 50 mg, about 100 mg, about 500 mg, about 600 mg, about 800 mg, about 1200 mg of a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib as an active ingredient, or a pharmaceutically acceptable salt, solvate, or prodrug thereof, as enteric-coated delayed-release tablets for oral administration. The pharmaceutical compositions further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

The pharmaceutical compositions provided herein may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredients) sufficient to produce the desired therapeutic effect, in association with the required pharmaceutical carriers or excipients. Examples of unit-dosage forms include ampules, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The compositions provided herein may be administered alone, or in combination with one or more other compounds provided herein, one or more other active ingredients. The pharmaceutical compositions that comprise a compound provided herein may be formulated in various dosage forms for oral, parenteral, buccal, intranasal, epidural, sublingual, pulmonary, local, rectal, transdermal, or topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified Release Drug Deliver Technology, Rathbone et al., Eds., Drugs and the Pharmaceutical Science, Marcel Dekker, Inc.: New York, N.Y., 2002; Vol. 126).

The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the administration of the combinations may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the combinations may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

As described herein, the compositions and methods for using the composition comprising a combination of a 1,2-diphenylpyrrole derivative and an EGFR inhibitor, may be formulated without carriers or excipients or may be combined with one or more pharmaceutically acceptable carriers for administration. For example, solvents, diluents and the like, and may be administered orally in such forms as tablets, capsules, dispersible powders, granules, or suspensions containing, for example, from about 0.05 to about 5% of suspending agent, syrups containing, for example, from about 10 to about 50% of sugar, and elixirs containing, for example, from about 20 to about 50% ethanol, and the like. Such pharmaceutical preparations may contain, for example, from about 0.05 up to about 90% of the active ingredient in combination with the carrier, more usually between about 5% and about 60% by weight. Also, the compositions and methods for using the composition comprising a combination of a 1,2-diphenylpyrrole derivative and an EGFR inhibitor wherein the 1,2-diphenylpyrrole derivative is 2-(4-ethoxyphenyl)-4-methyl-1-(4-sulfamoylphenyl)-pyrrole and the EGFR inhibitor is erlotinib, may be formulated without carriers or excipients or may be combined with one or more pharmaceutically acceptable carriers for administration.

The effective dosage of each active ingredient employed may vary depending on the particular compound employed, the mode of administration and the severity of the condition being treated. The projected daily dosage of the EGFR inhibitor will depend on its potency. For the purpose of comparison N-[4-[(3-bromophenyl)amino]-6-quinazolinyl]-2-butynamide has an IC₅₀ of 2 nM in the test procedure which measured the inhibition of the phosphorylation of the tyrosine residue of a peptide substrate catalyzed by EGFR kinase. Similarly, the dosage of the 1,2-diphenylpyrrole derivative inhibitor used depends on the relative potency of 1,2-diphenylpyrrole derivative inhibitor, compared for example to sulindac. Numerous methods for evaluating and comparing 1,2-diphenylpyrrole derivative inhibitor potency are known to one of skill in the art. In one embodiment, an oral daily dosage of the 1,2-diphenylpyrrole derivative inhibitor is in the range of about 0.01 to about 15 mg/kg, and the projected daily dosage of the EGFR inhibitor is in the range of about 0.3 to about 6 mg/kg. This dosage regimen may be adjusted to provide the optimal therapeutic response. For example, several divided doses may be administered daily or the dose may be proportionally reduced as indicated by the exigencies of the therapeutic situation. The 1,2-diphenylpyrrole derivative inhibitor and the EGFR inhibitor may also be administered as a combined dosage unit, or as separate components. When administered as separate components, each component may be administered at the same time, or at different times during the treatment period.

It is understood, however, that a specific dose level for any particular patient will depend upon a variety of factors such as, for example, decreases in the liver and kidney function.

Treatment dosages generally may be titrated to optimize safety and efficacy. Typically, dosage-effect relationships from in vitro studies initially can provide useful guidance on the proper doses for patient administration. Studies in animal models also generally may be used for guidance regarding effective dosages for treatment of cancers in accordance with the present disclosure. In terms of treatment protocols, it should be appreciated that the dosage to be administered will depend on several factors, including the particular agent that is administered, the route administered, the condition of the particular patient, etc. Determination of these parameters are well within the skill of the art. These considerations, as well as effective formulations and administration procedures are well known in the art and are described in standard textbooks.

### Oral Formulations

Oral formulations containing the active combinations described herein may comprise any conventionally used oral forms, including tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups, buccal forms, and oral liquids. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. In some embodiments are surface modifying agents which include nonionic and anionic surface modifying agents. For example, surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

### Oral Administration

As described herein, the combination regimen can be given simultaneously or can be given in a staggered regimen, with a 1,2-diphenylpyrrole derivative being given at a different time during the course of chemotherapy than an EGFR inhibitor. This time differential may range from several minutes, hours, days, weeks, or longer between administration of the two compounds. Therefore, the term combination does not necessarily mean administered at the same time or as a unitary dose, but that each of the components are administered during a desired treatment period. The agents may also be administered by different routes. As is typical for chemotherapeutic regimens, a course of chemotherapy may be repeated several weeks later, and may follow the same timeframe for administration of the two compounds, or may be modified based on patient response.

In other embodiments, the pharmaceutical compositions provided herein may be provided in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar, starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

In further embodiments, the pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

In other embodiments, the pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Miccellar dosage forms can be prepared as described in U.S. Pat. No. 6,350,458.

In other embodiments, the pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, sustained, pulsed-, controlled, targeted-, and programmed-release forms.

In further embodiments, the pharmaceutical compositions provided herein may be co-formulated with other active ingredients which do not impair the desired therapeutic action, or with substances that supplement the desired action, such as other cholinergic agents, other serotoninergic agents, alpha adrenergic agents, CCK_{A} antagonists, 5-HT₃ antagonists, NMDA receptor antagonists, opioids, prokinetics, tachykinins, antalarmin, and Z-338.

### Parenteral Administration

In some embodiments, the pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In other embodiments, the pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

In some embodiments, the pharmaceutical compositions provided herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### Modified Release

In other embodiments, the pharmaceutical compositions provided herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

### Matrix Controlled Release Devices

In some embodiments, the pharmaceutical compositions provided herein in a modified release dosage form may be fabricated using a matrix controlled release device known to those skilled in the art (see, Takada, et al in “Encyclopedia of Controlled Drug Delivery,” Vol. 2, Mathiowitz ed., Wiley, 1999).

In one embodiment, the pharmaceutical compositions provided herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylenelvinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate, and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

In other embodiments, the pharmaceutical compositions provided herein in a modified release dosage form may be prepared by methods known to those skilled in the art, including direct compression, dry or wet granulation followed by compression, melt-granulation followed by compression.

### 2. Osmotic Controlled Release Devices

In some embodiments, the pharmaceutical compositions provided herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol, organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semi-permeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semi-permeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semi-permeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semi-permeable membrane, the composition of the core, and the number, size, and position of the delivery ports.

The pharmaceutical compositions in an osmotic controlled-release dosage form may further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In other embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

In some embodiments, the pharmaceutical compositions provided herein in a modified release dosage form may be fabricated a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates may be made by the processes know to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein may be blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

### 4. Targeted Delivery

In some embodiments, the pharmaceutical compositions provided herein may also be formulated to be targeted to a particular tissue, receptor, or other area of the body of the subject to be treated, including liposome-, resealed erythrocyte-, and antibody-based delivery systems. Examples include, but are not limited to, U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874, all of which are incorporated herein by their entirety.

### Immediate Release

In some embodiments, the pharmaceutical compositions provided herein in an immediate release dosage form are capable of releasing not less than 75% of the therapeutically active ingredient or combination and/or meet the disintegration or dissolution requirements for immediate release tablets of the particular therapeutic agents or combination included in the tablet core, as set forth in USP XXII, 1990 (The United States Pharmacopeia.)

### Topical Administration

In other embodiments, the pharmaceutical compositions provided herein may be administered topically to the skin, orifices, or mucosa. The topical administration, as used herein, include (intra)dermal, conjuctival, intracorneal, intraocular, ophthalmic, auricular, transdermal, nasal, vaginal, uretheral, respiratory, and rectal administration.

In further embodiments, the pharmaceutical compositions provided herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein may also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffeting agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

In some embodiments, the pharmaceutical compositions may also be administered topically by electroporation, iontophoresis, phonophoresis, sonophoresis and microneedle or needle-free injection, such as POWDERJECTT™ (Chiron Corp., Emeryville, Calif.), and BIOJECT™ (Bioject Medical Technologies Inc., Tualatin, Oreg.).

The pharmaceutical compositions provided herein may be provided in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used Rectal and vaginal suppositories may be prepared by the compressed method or molding. The typical weight of a rectal and vaginal suppository is about 2 to about 3 g.

The pharmaceutical compositions provided herein may be administered ophthalmically in the forms of solutions, suspensions, ointments, emulsions, gel-forming solutions, powders for solutions, gels, ocular inserts, and implants.

The pharmaceutical compositions provided herein may be administered intranasally or by inhalation to the respiratory tract. The pharmaceutical compositions may be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be provided as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient provided herein, a propellant as solvent; and/or an surfactant, such as sorbitan trioleate, oleic acid, or an oligolactic acid.

In another embodiment, the pharmaceutical compositions provided herein may be micronized to a size suitable for delivery by inhalation, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

In one embodiment, the pharmaceutical compositions provided herein for topical administration may be formulated to be immediate release or modified release, including delayed-, sustained-, pulsed-, controlled-, targeted, and programmed release.

### EXAMPLES

### Example 1

### Synthesis of 2-(4-ethoxyphenyl)-4-methyl-1-(4-sulfamoylphenyl)-pyrrole

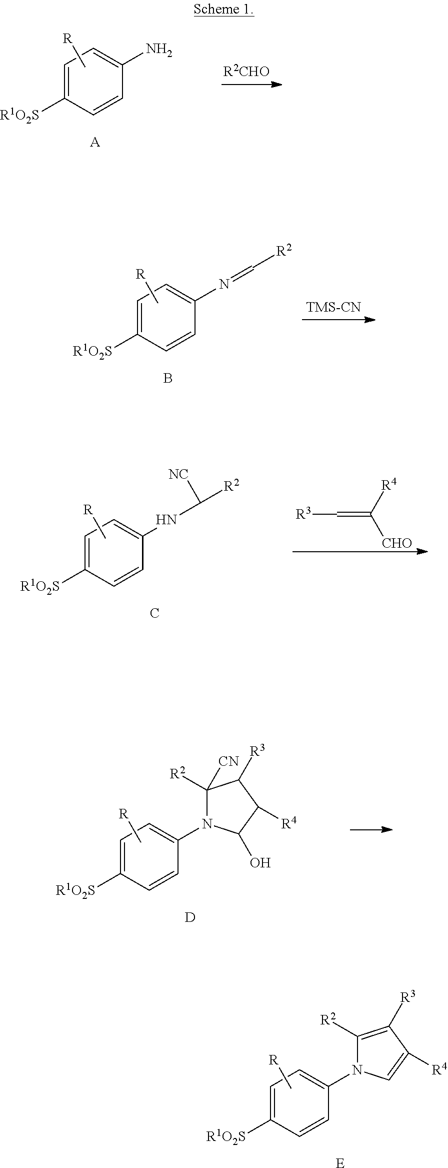

Substituted benzaldehyde undergoes dehydration condensation by reaction with aniline compound A in an inert solvent at a temperature of between 5° C. to 200° C. to give aldimine compound B. Trimethylsilyl cyanide is then reacted with aldimine compound B in the presence of a Lewis acid to afford anilinonitrile C. An α-β-unsaturated aldehyde is then reacted with anilinonitrile C to afford compound D which then undergoes dehydration and dehydrogencyanation under basic conditions in a modification of the method described in Ann. Chem. 589, 176 (1954).

### Example 2

### Synthesis of Erlotinib

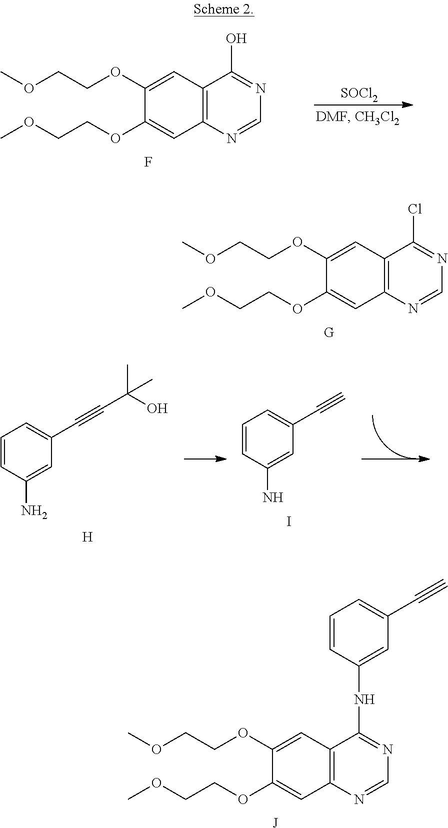

Starting material compound H is heated in a suspension of metal alkali and solvent, then heated to give compound I. Starting material alcohol F, is placed in a solvent mixture of thionyl chloride, methylene chloride and dimethylformamide to give the chloride G. Chloride G is then coupled with compound I to give erlotinib J.

### Example 3

### Pharmacokinetics Metabolism of 2-(4-ethoxyphenyl)-4-methyl-1-(4-sulfamoylphenyl)-pyrrole

Orally administered 2-(4-ethoxyphenyl)-4-methyl-1-(4-sulfamoylphenyl)-pyrrole is rapidly absorbed in all species examined (mice, rats, dogs, and monkeys). Peak plasma concentrations were achieved between 1 and 3 hours after a dose of 5 mg/kg. The elimination half life (t_{1/2}) was 4-5 hours in rodents and dogs, and approximately 2 hours in monkeys. Oral availability was greatest in rodent, and was reduced in dogs and monkeys (59 and 34% respectively).

### Example 4

### Toxicology of 2-(4-ethoxyphenyl)-4-methyl-1-(4-sulfamoylphenyl)-pyrrole

Toxicological evaluation of 2-(4-ethoxyphenyl)-4-methyl-1-(4-sulfamoylphenyl)-pyrrole in mice, rats, dogs and monkeys revealed expected findings related to inhibition of cyclooxygenase and consistent with animal safety observations with other COX-2 selective inhibitors. In single dose studies, the minimum lethal dose of 2-(4-ethoxyphenyl)-4-methyl-1-(4-sulfamoylphenyl)-pyrrole was 600 mg/kg in rats and >2000 mg/kg in dogs.

### Example 5

### Biological Evaluation

### COX-2 Selective Inhibitors

### HT-29 Model:

Mice are injected subcutaneously in the left paw (1×10⁶ tumor cells suspended in 30% Matrigel) and tumor volume is evaluated using a phlethysmometer twice a week for 30-60 days. Implantation of human colon cancer cells (HT-29) into nude mice produces tumors that will reach 0.6-2 ml between 30-50 days. Blood is drawn twice during the experiment in a 24 h protocol to assess plasma concentration and total exposure by AUC analysis. The data is expressed as the mean+/−SEM. Student's and Mann-Whitney tests are used to assess differences between means using the InStat software package.

A. Mice injected with HT-29 cancer cells are treated with cytoxin i.p at doses of 50 mg/kg on days 5, 7 and 9 in the presence or absence of a composition comprising a combination of 2-(4-ethoxyphenyl)-4-methyl-1-(4-sulfamoylphenyl)-pyrrole with erlotinib in the diet. The efficacy of both agents are determined by measuring tumor volume. The results from these studies may demonstrate that a composition comprising a combination of 2-(4-ethoxyphenyl)-4-methyl-1-(4-sulfamoylphenyl)-pyrrole with erlotinib administered in the diet to tumor bearing mice can delay the growth of tumors and metastasis when administered as sole therapy.

B. In a second assay, mice are injected with HT-29 cancer cells are then treated with 5-FU on days 12 through 15. Mice injected with HT-29 cancer cells are treated with 5-FU i.p at doses of 50 mg/kg on days 12, 13, 14, and 15 in the presence or absence of a composition comprising a combination of 2-(4-ethoxyphenyl)-4-methyl-1-(4-sulfamoylphenyl)-pyrrole with erlotinib in the diet. The efficacy of both agents are determined by measuring tumor volume. Treatment using the composition may reduce tumor volume by up to 70%. In the same assay, 5-FU decreases tumor volume by 61%. Further, the composition and 5-FU may decrease tumor volume by 83%.

C. In a third assay, mice injected with HT-29 colon cancer cells are treated with 5-FU i.p 50 mg/kg on days 14 through 17 in the presence or absence of a composition comprising a combination of 2-(4-ethoxyphenyl)-4-methyl-1-(4-sulfamoylphenyl)-pyrrole with erlotinib (1600 ppm) and valdecoxib (160 ppm) in the diet. The efficacy of both agents are determined by measuring tumor volume. Treatment with 5-FU may result in a 35% reduction in tumor volume. Treatment with the composition and valdecoxib may reduce tumor volume by 52% and 69%, respectively. In the same assay, the combination of 5-FU and the composition may decrease tumor volume by 72% while the combination of 5-FU and valdecoxib may decrease tumor volume by 74%.

### Example 6

### In Vitro Inhibition of EGFR Kinase Activity

The in vitro activity of the combinations described herein in inhibiting the receptor tyrosine kinase may be determined by the following procedure. The activity of the combinations of the present disclosure, in vitro, can be determined by the amount of inhibition of the phosphorylation of an exogenous substrate (e.g., Lys₃—Gastrin or polyGluTyr (4:1) random copolymer (Posner et al., J. Biol. Chem., 1992, 267 (29), 20638-472)) on tyrosine by epidermal growth factor receptor kinase by a test compound relative to a control. Affinity purified, soluble human EGF receptor (96 ng) is obtained according to the procedure in G. N. Gill, W. Weber, Methods in Enzymology, 1987, 146, 82-8 from A431 cells (American Type Culture Collection, Rockville, Md.) and preincubated in a microfuge tube with EGF (2 μg/ml) in phosphorylation buffer+vanadate (PBV: 50 mM HEPES, pH 7.4; 125 mM NaCl; 24 mM MgCl₂; 100 μM sodium orthovanadate), in a total volume of 10 μl, for 20-30 minutes at room temperature. The composition comprising a combination of 2-(4-ethoxyphenyl)-4-methyl-1-(4-sulfamoylphenyl)-pyrrole with erlotinib, dissolved in dimethylsulfoxide (DMSO), is diluted in PBV, and 10 μl is mixed with the EGF receptor/EGF mix, and incubated for 10-30 minutes at 30° C. The phosphorylation reaction is initiated by addition of 20 μl ³³P-ATP/substrate mix (120 μM Lys₃-Gastrin (sequence in single letter code for amino acids, KKKGPWLEEEEEAYGWLDF), 50 mM Hepes pH 7.4, 40 μM ATP, 2 pCi .gamma.-[³³P]-ATP) to the EGFr/EGF mix and incubated for 20 minutes at room temperature. The reaction is stopped by addition of 10 μl stop solution (0.5 M EDTA, pH 8; 2 mM ATP) and 6 μl 2N HCl. The tubes are centrifuged at 14,000 RPM, 4° C., for 10 minutes. 35 μl of supernatant from each tube is pipetted onto a 2.5 cm circle of Whatman P81 paper, bulk washed four times in 5% acetic acid, 1 liter per wash, and then air dried. This results in the binding of substrate to the paper with loss of free ATP on washing. The [³³P] incorporated is measured by liquid scintillation counting. Incorporation in the absence of substrate (e.g., lys₃-gastrin) is subtracted from all values as a background and percent inhibition is calculated relative to controls without the composition present. Such assays, carried out with a range of doses of test combinations, allow the determination of an approximate IC₅₀ value for the in vitro inhibition of EGFR kinase activity. Other methods for determining the activity of the combinations presented herein are described in U.S. Pat. No. 5,747,498, the disclosure of which is incorporated herein.

### Example 7

### Pharmaceutical Compositions and Dosage Forms

Dosage formulations comprising pharmaceutical excipients and carriers and a pharmaceutical composition comprising a combination of erlotinib (A) and 2-(4-ethoxyphenyl)-4-methyl-1-(4-sulfamoylphenyl)-pyrrole (B) include:

| | Amount of A | |
|---|---|---|
| Combination | per tablet (mg) | Amount of B per tablet (mg) |
| A/B | 25 | 1, 5, 10, 25, 50, 100, 200, 300,400, |
| | | 600, 800, 1000, 1200 |
| A/B | 100 | 1, 5, 10, 25, 50, 100, 200, 300,400, |
| | | 600, 800, 1000, 1200 |
| A/B | 150 | 1, 5, 10, 25, 50, 100, 200, 300,400, |
| | | 600, 800, 1000, 1200 |
| A/B | 200 | 1, 5, 10, 25, 50, 100, 200, 300,400, |
| | | 600, 800, 1000, 1200 |
| A/B | 300 | 1, 5, 10, 25, 50, 100, 200, 300,400, |
| | | 600, 800, 1000, 1200 |
| A/B | 450 | 1, 5, 10, 25, 50, 100, 200, 300,400, |
| | | 600, 800, 1000, 1200 |

Dosage formulations described herein, including the formulations set forth in the above table, may be administered in a single fixed dose comprising a combination of 2-(4-ethoxyphenyl)-4-methyl-1-(4-sulfamoylphenyl)-pyrrole and erlotinib or as a separate administration of a single dose of 2-(4-ethoxyphenyl)-4-methyl-1-(4-sulfamoylphenyl)-pyrrole and a single dose of erlotinib.

### Example 8

### Treatment of Non-Small Cell Lung Cancer

A method for treating a subject having non-small cell lung cancer comprising administering to the subject a therapeutically effective amount of a combination comprising 2-(4-ethoxyphenyl)-4-methyl-1-(4-sulfamoylphenyl)-pyrrole and erlotinib or their respective pharmaceutically acceptable salt, solvate or prodrug is contemplated. The subject is treated using a single dosage form of a combination of 2-(4-ethoxyphenyl)-4-methyl-1-(4-sulfamoylphenyl)-pyrrole in about 100, 200, 300, 400, 600 about 800, and about 1000 and 1200 mg aliquots and erlotinib in about a 150 mg aliquot. The treatment of the subject using a separate administration of 2-(4-ethoxyphenyl)-4-methyl-1-(4-sulfamoylphenyl)-pyrrole in about 200, about 400, and about 800 mg aliquots and erlotinib in about a 150 mg aliquot is also contemplated. The maximum tolerated dose of this combination will be an endpoint for this study.

### Example 9

### Treatment of Breast Cancer

A method for treating a subject having breast cancer comprising administering to the subject a therapeutically effective amount of a combination comprising 2-(4-ethoxyphenyl)-4-methyl-1-(4-sulfamoylphenyl)-pyrrole and erlotinib or their respective pharmaceutically acceptable salt, solvate or prodrug is contemplated. Combined treatment with erlotinib and 2-(4-ethoxyphenyl)-4-methyl-1-(4-sulfamoylphenyl)-pyrrole is expected to achieve increased tumor inhibition compared with erlotinib administered as a single agent.

### Example 10

### Treatment of Colorectal Cancer

A method for treating a subject having colorectal cancer comprising administering to the subject a therapeutically effective amount of a combination comprising 2-(4-ethoxyphenyl)-4-methyl-1-(4-sulfamoylphenyl)-pyrrole and erlotinib or their respective pharmaceutically acceptable salt, solvate or prodrug is contemplated. Combined treatment with erlotinib and 2-(4-ethoxyphenyl)-4-methyl-1-(4-sulfamoylphenyl)-pyrrole are expected to achieve significantly increased tumor inhibition compared with either 2-(4-ethoxyphenyl)-4-methyl-1-(4-sulfamoylphenyl)-pyrrole or erlotinib administered as single agents.

### Example 11

### Treatment of Glioma

A method for treating a subject having glioma comprising administering to the subject a therapeutically effective amount of a combination comprising 2-(4-ethoxyphenyl)-4-methyl-1-(4-sulfamoylphenyl)-pyrrole and erlotinib or their respective pharmaceutically acceptable salt, solvate or prodrug is contemplated. The subject is treated using a single dosage form of a combination of 2-(4-ethoxyphenyl)-4-methyl-1-(4-sulfamoylphenyl)-pyrrole in about 1, about 5, about 10, about 25, about 50, about 100, about 200, about 300, about 400, about 600, about 800, about 1000, and about 1200 mg aliquots and erlotinib in about a 25 mg aliquot. The treatment of the subject using a separate administration of 2-(4-ethoxyphenyl)-4-methyl-1-(4-sulfamoylphenyl)-pyrrole in about 1, about 5, about 10, about 25, about 50, about 100, about 200, about 300, about 400, about 600, about 800, about 1000, and about 1200 mg aliquots and erlotinib in about 100, 150, 200, 300, or 450 mg aliquot is also contemplated. The treatment will also be administered in subjects with 2″ temozolomide failures.

### Example 12

### Treatment of Head and Neck Cancer

A method for treating a subject having head and neck cancer comprising administering to the subject a therapeutically effective amount of a combination comprising 2-(4-ethoxyphenyl)-4-methyl-1-(4-sulfamoylphenyl)-pyrrole and erlotinib or their respective pharmaceutically acceptable salt, solvate or prodrug is contemplated. The subject is treated using a single dosage form of a combination of 2-(4-ethoxyphenyl)-4-methyl-1-(4-sulfamoylphenyl)-pyrrole in about 1, about 5, about 10, about 25, about 50, about 100, about 200, about 300, about 400, about 600, about 800, about 1000, and about 1200 mg aliquots and erlotinib in about a 25 mg aliquot. The treatment of the subject using a separate administration of 2-(4-ethoxyphenyl)-4-methyl-1-(4-sulfamoylphenyl)-pyrrole in about 1, about 5, about 10, about 25, about 50, about 100, about 200, about 300, about 400, about 600, about 800, about 1000, and about 1200 mg aliquots and erlotinib in about 100, 150, 200, 300, or 450 mg aliquot is also contemplated. The treatment will also be administered in conjunction with one or one or more chemotherapy agents (e.g., paclitaxel [Taxol®], docetaxel [Taxotere®], gemcitabine [Gemzar®], doxorubicin [Doxil®]) which may be further combined with established chemotherapeutic agents (e.g., methotrexate [Trexall®, Methotrex®]). In another embodiment, therapy for the treatment of head and neck cancer according to the invention utilizes a combination of therapeutically effective amount of a composition comprising a combination of 2-(4-ethoxyphenyl)-4-methyl-1-(4-sulfamoylphenyl)-pyrrole and erlotinib and Erbitux®.

### Example 13

### Treatment of NSCLC

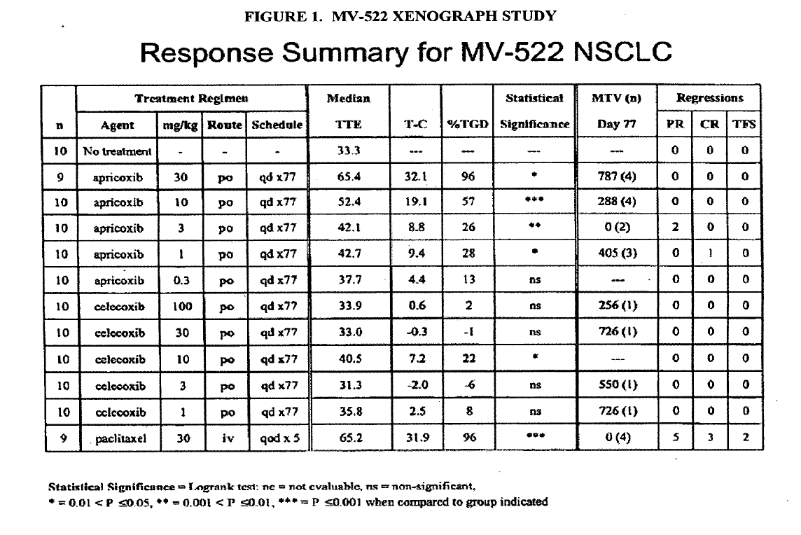

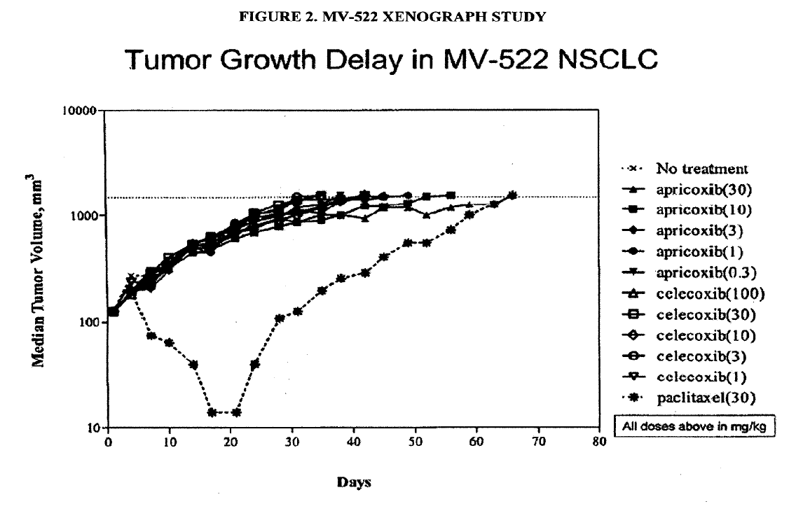

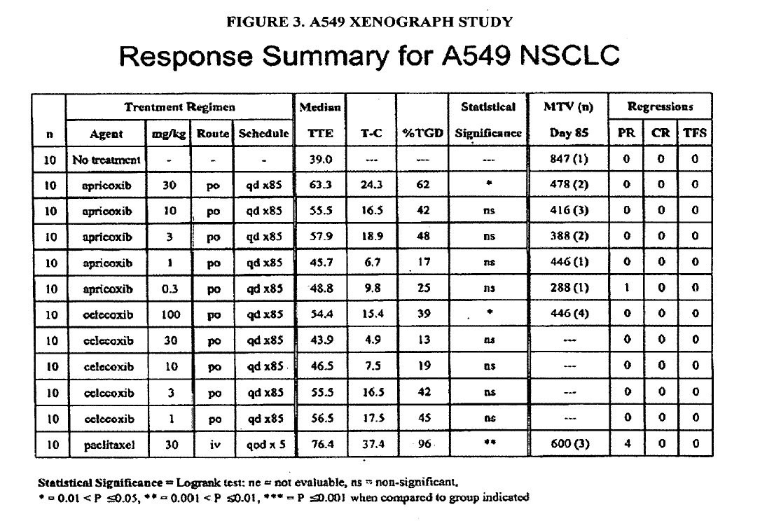

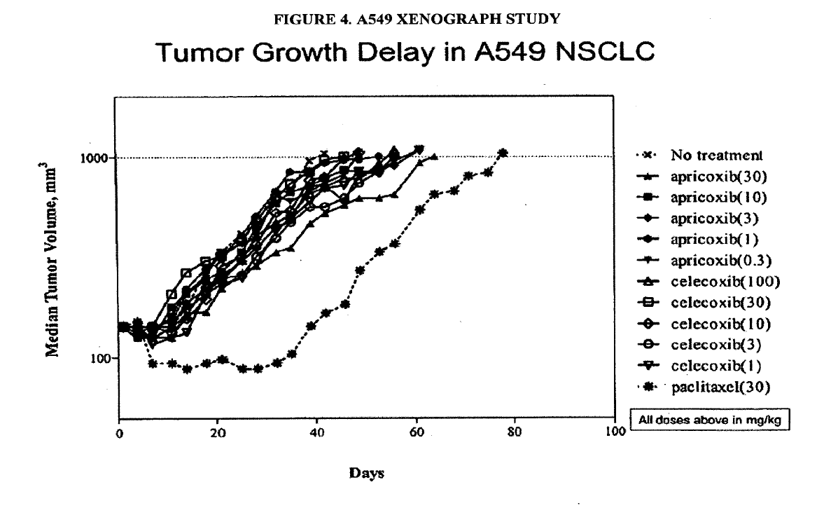

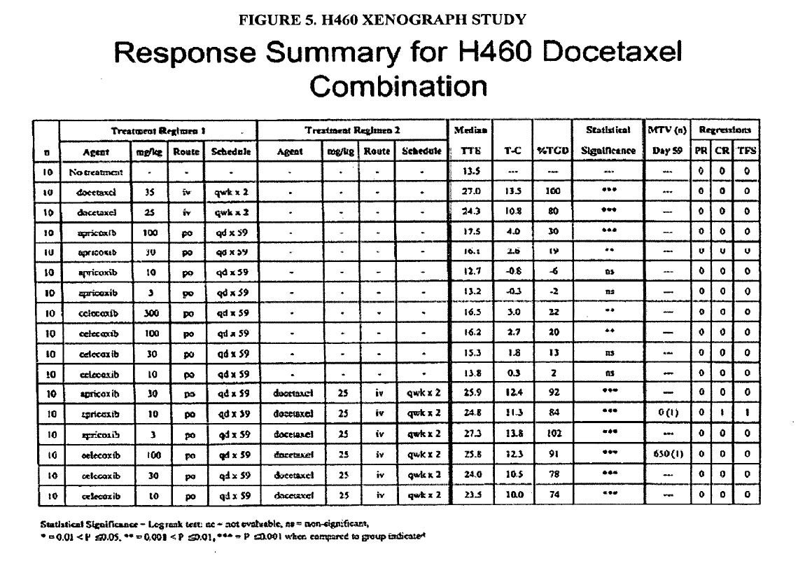

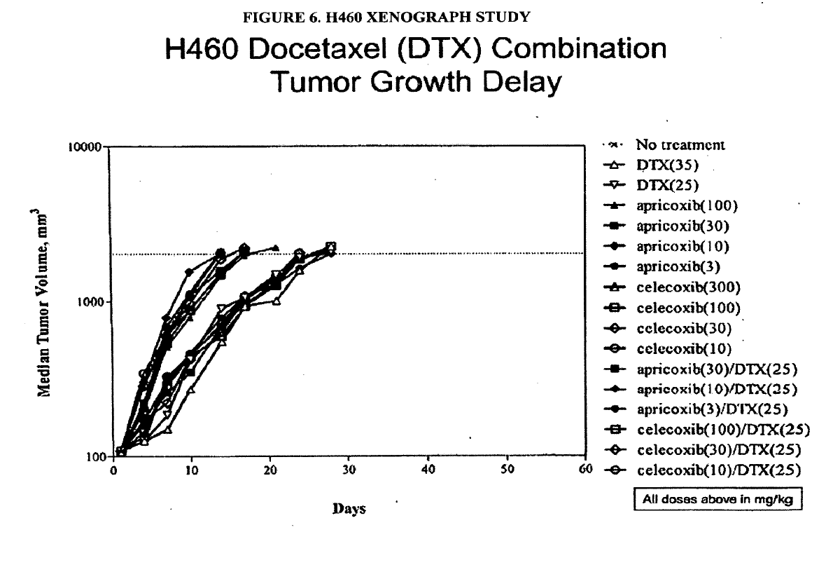

Activity of 2-(4-ethoxyphenyl)-4-methyl-1-(4-sulfamoylphenyl)-pyrrole, a selective COX-2 inhibitor, alone and in combination with docetaxel in human non-small cell lung carcinoma xenografts. 2-(4-ethoxyphenyl)-4-methyl-1-(4-sulfamoylphenyl)-pyrrole was administered orally on a continuous daily regimen in female athymic nude mice bearing established sc-implanted xenografts. The models evaluated included MV522, A549 and H460 NSCLC. In each tumor model, 2-(4-ethoxyphenyl)-4-methyl-1-(4-sulfamoylphenyl)-pyrrole was compared over a broad dosage range with celecoxib. Monotherapy was evaluated in the MV-522 (see FIGS. 1 and 2) and the A549 cell line (see FIGS. 3 and 4), and combinations of the COX-2 inhibitors with docetaxel were evaluated in the H460 model (see FIGS. 5 and 6), which was chosen for its moderate sensitivity to taxanes. The experiments used a tumor growth delay (TGD) endpoint based on median time to endpoint, i.e., tumor volume of 1000-2000 mm³, and treatments were compared to untreated controls using the Logrank test for statistical significance.

In the slow-growing MV522 model, 2-(4-ethoxyphenyl)-4-methyl-1-(4-sulfamoylphenyl)-pyrrole at its MTD of 30 mg/kg produced a significant 32.1-day TGD (96%, p=0.030). There was also a significant TGD of 19.1 days at 10 mg/kg of TG01 (57%, p=0.0002). In contrast, celecoxib did not produce dose-dependent TGD in MV522 NSCLC.

2-(4-Ethoxyphenyl)-4-methyl-1-(4-sulfamoylphenyl)-pyrrole also had significant activity in the slow-growing A549 NSCLC with a 24.3-day TGD (62%, p=0.030) at its MTD. In this model, celecoxib also showed a significant but smaller TGD of 15.4 days (39%, p=0.035) at 100 mg/kg.

In the fast-growing H460 NSCLC, both 2-(4-ethoxyphenyl)-4-methyl-1-(4-sulfamoylphenyl)-pyrrole and celecoxib produced a minimal, albeit statistically significant, TGD of 4.0 days (30%, p=0.0042) and 3.0 days (22%, p=0.0080), respectively. Docetaxel was active with a TGD of 10.8 days (80%, p<0.0001) at 25 mg/kg (iv weekly ×3). Combinations of docetaxel with 2-(4-ethoxyphenyl)-4-methyl-1-(4-sulfamoylphenyl)-pyrrole or celecoxib were not significantly different from docetaxel alone with TGD values of 12.4 (92%, p=0.99) and 12.3 (91%, p=0.85) days, respectively.

2-(4-Ethoxyphenyl)-4-methyl-1-(4-sulfamoylphenyl)-pyrrole had statistically significant activity in all 3 tumor models evaluated, although the activity in the fast-growing H460 NSCLC was minimal 2-(4-ethoxyphenyl)-4-methyl-1-(4-sulfamoylphenyl)-pyrrole had greater activity than celecoxib in MV522 and A549 NSCLC.

### Example 14

### A Randomized, Double-Blind, Placebo-Controlled Multicenter Phase 2 Study of the Efficacy and Safety of Apricoxib in Combination with Either Docetaxel or Pemetrexed in Non-Small Cell Lung Cancer Patients

This clinical trial is a phase 2, randomized, double-blind, placebo-controlled, multicenter study with an unblinded run-in period.

Patients undergo screening assessments and the 5-day run-in during the same 14-day period. It is permitted to conduct the apricoxib run-in in anticipation of completion of eligibility requirements (i.e. obtaining CT scans etc) as neither toxic nor therapeutic effects during screening are likely. Patients must meet eligibility requirements for history, physical, performance status, and laboratory parameters prior to beginning the screening phase. Patients are monitored closely during this period for toxicity. Prior to beginning the 5-day run-in period, patients discontinue aspirin for 7 days and other NSAIDs for 2 days. Patients may undergo urine screening and obtain needed scans during the same time period. During the unblended run-in phase, patients are given apricoxib 400 mg QD for 5 days. Urinary PGE-M is measured on the first day and last day of the run-in period. There is a +2-day window for the assessments and collection of the urinary PGE-M on Day 5 of the run-in period. Patients are selected for randomization based on at least a 50% decrease from their baseline PGE-M level after a 5-day run in of single agent apricoxib. There is a minimum of a 48-hour wash-out period of apricoxib between the end of the run-in period and the start of double-blind treatment (Cycle 1, Day 1).

On Day 1 of Cycle 1, eligible patients are randomized in a 1:1 ratio to treatment with apricoxib (AP) and docetaxel (DC) or placebo (P) and docetaxel (DC); or apricoxib (AP) and pemetrexed (PE) or placebo (P) and pemetrexed (PE). Patients randomized to AP/DC or AP/PE receive apricoxib tablets. Patients randomized to P/DC or P/PE receive placebo tablets that match apricoxib tablets. Each cycle of study treatment is 21 days. Upon completion of each study cycle without tumor progression or intolerable toxicity, patients start a new cycle of study treatment. Study treatment continue as determined by the Investigator until disease progression, intolerable AEs; death, voluntary withdrawal from the study by the patient, or discontinuation of the study by the sponsor.

During the first cycle of study treatment, safety and tolerability assessments (spontaneous reports of AEs, physical examination, vital signs, ECOG PS, clinical laboratory tests) are measured weekly on Days 1, 8, and 15. During subsequent cycles, safety and tolerability assessments are measured every 21 days on Day 1. There is a ±3-day window for all post-randomization study visits.

Tumor assessments (magnetic resonance imaging [MRI], conventional or spiral computed tomography [CT], x-rays) are performed at baseline, at the end of every even-numbered cycle (e.g., Day 1 of Cycles 3, 5, etc.), and at the end of study (EOS)/early termination (unless they have been performed within 14 days of this visit). Other efficacy assessments (physical examinations, Faces Pain Scale, Functional Assessment of Lung Cancer Therapy [FACT-L]) are performed weekly during Cycle 1, on Day 1 of subsequent cycles, and at EOS/early termination.

Tumor COX-2 IHC is assessed on a paraffin-embedded tumor sample from a previous biopsy. A minimum of 5 slides are collected. The pharmacodynamic tests are run at a later date (except for the urinary PGE-M). Other pharmacodynamic samples are obtained during screening and on Day 1 of Cycle 2. Urinary PGE-M and COX-2 IHC are measured. Other pharmacodynamic assessments include plasma for deoxyribonucleic acid (DNA) gene analysis, ribonucleic acid (RNA) gene expression analysis, proteomics, and drug level evaluation. The following genes/proteins may be evaluated; however, this list is not exhaustive: CD44, MMp2, Zeb1, Snail, IL-10, IL-12, FOXP3, CXCL5, CXCL8, VEGF, Survivin, IGF-BP-3, and IL-6. These genes/proteins evaluate COX-2 mechanisms involved with tumor invasion, immune regulation, angiogenesis, and apoptosis. Within 3 days of discontinuation of study treatment, patients attend an EOS/early termination visit, where efficacy and safety assessments is performed.

Patient compliance with the treatment regimen is assessed by patient dosing calendars. The date and time study treatment is taken is recorded by patients. In addition, dose delays and reductions are recorded by study personnel and included on the case report forms (CRFs). All instances of noncompliance and all resulting protocol deviations are recorded on the CRFs.

1. A method for treating a subject having cancer, comprising administering to the subject, a therapeutically effective amount of a combination comprising a 1,2-diphenylpyrrole derivative or the respective pharmaceutically acceptable salt, solvate, polymorph or prodrug and a microtubule inhibitor.

2. The method of claim 1 wherein the 1,2-diphenylpyrrole derivative has the following formula: 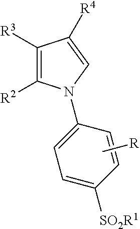
wherein:
R is a hydrogen atom, a halogen atom or an alkyl group having from 1 to 6 carbon atoms;
R¹ is an alkyl group having from 1 to 6 carbon atoms or an amino group;
R² is a phenyl group which is unsubstituted or is substituted by at least one substituent selected from the group consisting of substituents α and substituents β;
R³ is a hydrogen atom, a halogen atom or an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms;
R⁴ is a hydrogen atom; an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms; a cycloalkyl group having from 3 to 8 carbon atoms, an aryl group; or an aralkyl group; said aryl group having from 6 to 14 ring carbon atoms in a carbocyclic ring and are unsubstituted or are substituted by at least one substituent selected from the group consisting of substituents α and substituents β;
said aralkyl group are an alkyl group having from 1 to 6 carbon atoms and which are substituted by at least one aryl group as defined above;
said substituents α are selected from the group consisting of a hydroxy group, a halogen atom, an alkoxy group having from 1 to 6 carbon atoms and an alkylthio group having from 1 to 6 carbon atoms;
said substituents β are selected from the group consisting of an alkyl group which has from 1 to 6 carbon atoms and which is unsubstituted or are substituted by at least one substituent selected from the group consisting of a hydroxy group, a halogen atom, an alkoxy group having from 1 to 6 carbon atoms and an alkylthio group having from 1 to 6 carbon atoms; an alkanoyloxy group having from 1 to 6 carbon atoms; a mercapto group; an alkanoylthio group having from 1 to 6 carbon atoms; an alkylsulfinyl group having from 1 to 6 carbon atoms; a cycloalkloxy group having from 3 to 8 carbon atoms; a haloalkoxy group having from 1 to 6 carbon atoms; and an alkylenedioxy group having from 1 to 6 carbon atoms; or a pharmaceutically acceptable salt, solvate, or prodrug.

3. The method of claim 2 wherein:
R is a hydrogen atom, a halogen atom or an alkyl group having from 1 to 4 carbon atoms;
R¹ is a methyl group or an amino group;
R² is an unsubstituted phenyl group or a phenyl group which is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and which is substituted by at least one substituent selected from the group consisting of a halogen atom, an alkoxy group having from 1 to 4 carbon atoms and an alkylthio group having from 1 to 4 carbon atoms; a haloalkoxy group having from 1 to 4 carbon atoms; and an alkylenedioxy group having from 1 to 4 carbon atoms;
R³ is a hydrogen atom, a halogen atom, an unsubstituted alkyl group having from 1 to 4 carbon atoms or a substituted alkyl group having from 1 to 4 carbon atoms and substituted by at least one substituent selected from the group consisting of a halogen atom, an alkoxy group having from 1 to 4 carbon atoms and an alkylthio group having from 1 to 4 carbon atoms;
R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to carbon atoms; a cycloalkyl group having from 3 to 6 carbon atoms; an aryl group which has from 6 to 10 ring carbon atoms and which is unsubstituted or is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to 4 carbon atoms; and a cycloalkyloxy group having from 3 to 7 carbon atoms; an aralkyl group having from 1 to 4 carbon atoms in the alkyl part and containing at least one said aryl group; or a pharmaceutically acceptable salt, solvate, or prodrug.

4. The method of claim 3 wherein:
R is a hydrogen atom;
R¹ is an amino group;
R² is an unsubstituted phenyl group or a phenyl group which is substituted by at least one substituent selected from the group consisting of a halogen atom, an alkoxy group having from 1 to 4 carbon atoms, an alkylthio group having from 1 to 4 carbon atoms, an alkyl group having from 1 to 4 carbon atoms, a haloalkyl group having from 1 to 4 carbon atoms, a haloalkoxy group having from 1 to 4 carbon atoms and a alkylenedioxy group having from 1 to 4 carbon atoms;
R³ is a hydrogen atom, a halogen atom, an alkyl group having from 1 to 4 carbon atoms or a haloalkyl group having from 1 to 4 carbon atoms;
R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group and an alkoxy group having from 1 to 4 carbon atoms; a cycloalkyl group having from 3 to 6 carbon atoms; an aryl group which has from 6 to 10 ring carbon atoms and which is unsubstituted or is substituted by at least one substituent selected from the group consisting of a hydroxy group; a halogen atom; an alkoxy group having from 1 to 4 carbon atoms; an unsubstituted alkyl group having from 1 to 4 carbon atoms; an alkyl group having from 1 to 4 carbon atoms and which is unsubstituted or substituted by at least one halogen atom; and a cycloalkyloxy group having from 3 to 7 carbon atoms; and an aralkyl group having from 1 to 4 carbon atoms in the alkyl part and containing at least one said aryl group; or a pharmaceutically acceptable salt, solvate, or prodrug.

5. The method of claim 4 wherein the 1,2-diphenylpyrrole derivative is selected from the group consisting of: 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole.

6. The method of claim 5 wherein the 1,2-diphenylpyrrole derivative is 2-(4-ethoxyphenyl)-4-methyl-1-(4-sulfamoylphenyl)-pyrrole.

7. The method of claim 1 wherein the microtubule inhibitor is selected from docetaxel, paclitaxel and ixabepilone.

8. The method of claim 1 wherein the microtubule inhibitor is docetaxel.

9. The method of claim 1 wherein the 1,2-diphenylpyrrole derivative is 2-(4-ethoxyphenyl)-4-methyl-1-(4-sulfamoylphenyl)-pyrrole and the microtubule inhibitor is docetaxel.

10. The method of claim 1 wherein the 1,2-diphenylpyrrole derivative and the microtubule inhibitor are administered sequentially in either order or simultaneously.

11. The method of claim 1 wherein the 1,2-diphenylpyrrole derivative is administered first.

12. The method of claim 1 wherein the microtubule inhibitor is administered first.

13. The method of claim 1 wherein administering the combination enhances treatment of the subject.

14. The method of claim 1 wherein administering the combination reduces the side effects of the treatment of cancer compared to a treatment with the microtubule inhibitor alone or a treatment of the 1,2-diphenylpyrrole derivative alone.

15. The method of claim 1 wherein administering the combination is through oral, parenteral, buccal, intranasal, epidural, sublingual, pulmonary, local, rectal, or transdermal administration.

16. The method of claim 15 wherein administering the combination is through parenteral administration.

17. The method of claim 16 wherein parenteral administration is intravenous, subcutaneous, intrathecal, or intramuscular.

18. The method of claim 1 wherein the 1,2-diphenylpyrrole derivative is administered orally every day and the microtubule inhibitor is administered by injection with a frequency selected from once every day, once every other day, once every seven days, once every fourteen days, once every twenty-one days, and once every twenty-eight days per a treatment cycle.

19. The method of claim 1 wherein the cancer is selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

20. The method of claim 19 wherein the cancer is non-small cell lung cancer, pancreatic cancer, breast cancer, ovarian cancer, colorectal cancer, and head and neck cancer.

21-97. (canceled)
